SEQUENCE LISTING (19 sequences)

SEQ ID NO: 1 - protein, 809 aa, Artificial Sequence
  Description of Artificial Sequence Synthetic polypeptide
MNIKKEFIKVISMSCLVTAITLSGPVFIPLVQGAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLARYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 2 - protein, 33 aa, Artificial Sequence
  Description of Artificial Sequence Synthetic polypeptide
MNIKKEFIKVISMSCLVTAITLSGPVFIPLVQG

SEQ ID NO: 3 - protein, 776 aa, Artificial Sequence
  Description of Artificial Sequence Synthetic polypeptide
AGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLARYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 4 - protein, 9 aa, Human papillomavirus
RAHYNIVTF

SEQ ID NO: 5 - protein, 10 aa, Homo sapiens
FYQLAKTCPV

SEQ ID NO: 6 - protein, 15 aa, Human immunodeficiency virus
RIQRGPGRAFVTIGK

SEQ ID NO: 7 - protein, 9 aa, Unknown
  Description of Unknown MAGE-1 peptide
SAYGEPRKL

SEQ ID NO: 8 - protein, 9 aa, Unknown
  Description of Unknown MAGE-3 peptide
FLWGPRALV

SEQ ID NO: 9 - protein, 9 aa, Homo sapiens
MLLAVLYCL

SEQ ID NO: 10 - protein, 9 aa, Homo sapiens
YMNGTMSQV

SEQ ID NO: 11 - protein, 9 aa, Unknown
  Description of Unknown Listeriolysin-O peptide
GYKDGNEYI

SEQ ID NO: 12 - protein, 9 aa, Unknown
  Description of Unknown P60 peptide
KYGVSVQDI

SEQ ID NO: 13 - protein, 12 aa, Unknown
  Description of Unknown MART-1 M-9 peptide
AAAAAGIGILTV

SEQ ID NO: 14 - protein, 10 aa, Unknown
  Description of Unknown MART-1 M10-3 peptide
EAAGIGILTV

SEQ ID NO: 15 - protein, 9 aa, Unknown
  Description of Unknown BAGE-1 peptide
AARAVFLAL

SEQ ID NO: 16 - protein, 9 aa, Unknown
  Description of Unknown P1A peptide
LPYLGWLVF

SEQ ID NO: 17 - protein, 8 aa, Unknown
  Description of Unknown Connexin gap junction derived Mut 1 peptide
FEQNTAQP

SEQ ID NO: 18 - protein, 8 aa, Unknown
  Description of Unknown Connexin gap junction derived Mut 2 peptide
FEQNTAQA

SEQ ID NO: 19 - protein, 8 aa, Artificial Sequence
  Description of Artificial Sequence Synthetic peptide
SIINFEKL

(19) United States
(12) Patent Application Publication
(10) Pub. No.: US 2018/0117144 A1
(43) Pub. Date: May 3, 2018

(54) TARGETED CYTOSOLIC DELIVERY OF ANTIGENIC COMPOUNDS

(71) Applicant: President and Fellows of Harvard College, Cambridge, MA (US)

(72) Inventors: Andrew J. MCCLUSKEY, Shrewsbury, MA (US); R. John COLLIER, Wellesley, MA (US); Catarina V. NOGUEIRA, Somerville, MA (US); Michael STARNBACH, Needham, MA (US)

(73) Assignee: President and Fellows of Harvard College, Cambridge, MA (US)

(21) Appl. No.: 15/565,752

(22) PCT Filed: Apr. 11, 2016
(86) PCT No.: PCT/US2016/026902

§ 371 Date: Oct. 11, 2017

Related U.S. Application Data
(60) Provisional application No. 62/146,652, filed on Apr. 13, 2015.

(51) Int. Cl. A61K 39/385

(52) U.S. Cl. CPC A61K 39/385; A61K 2039/572

(57) ABSTRACT

The invention relates to engineered anthrax toxin compositions that can target antigen-presenting cells such as dendritic cells. Specifically, the compositions comprise (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least one repeat of a disease-specific antigen. The invention also relates to methods of using these compositions for targeted delivery to dendritic cells, methods of enhancing CTL activation, and methods of inducing an immune response to cancers, bacteria, and/or viruses.

### CROSS-REFERENCE TO RELATED APPLICATIONS

This application claims benefit under 35 U.S.C. § 119(e) of U.S. Provisional Application No. 62/146,652 filed Apr. 13, 2015, the contents of which are incorporated herein by reference in their entirety.

### GOVERNMENT SUPPORT

This invention was made with government support under grant Nos. AI039558, AI062827, AI097691 awarded by the National Institutes of Health. The government has certain rights in the invention.

### TECHNICAL FIELD

The present invention relates to engineered anthrax toxin components that can target dendritic cells, methods of delivering compounds to the cytosol of dendritic cells, methods of immunization, and methods of enhancing cytotoxic-T lymphocyte activation.

### BACKGROUND

Protective immunity, mediated by cytotoxic-T lymphocytes (CTL, also known as CD8⁺T cells), is important for efficient clearance of intracellular pathogens and effective immunization against many cancers. CTLs recognize foreign antigenic proteins in the cytoplasm of host cells and target those cells for destruction. To activate protective CTL-mediated immunity and to generate long-term memory against a certain pathogen, antigens derived from the pathogen must be delivered to the cytosol of host cells in vivo. Inefficient antigen delivery has contributed to a lag in vaccine development, highlighting the need for improved strategies to enhance cytosolic delivery of antigen to the appropriate cell type to generate robust CTL activation.

Targeting delivery of disease-specific antigens to antigen-presenting cells (APC), for example dendritic cells (DC), has emerged as an attractive strategy to improve CTL activation. Although targeted approaches are an improvement over free antigens, they achieve only modest results likely due to the inability of antigens to cross the endosomal membrane. There is an unmet need to develop methods of vaccination that target disease-specific antigens to DCs and achieve increased cytosolic delivery and robust CTL activation, without resorting to the use of live infectious organisms or recombinant DNA vectors.

### SUMMARY

The invention is based, at least in part, on the discovery that engineered anthrax toxin (ATx) systems can be used to deliver antigens into antigen-presenting cells such as dendritic cells, which allow enhanced CTL activation. Accordingly, in some aspects and embodiments, the invention relates to methods of delivering compounds to the cytosol of a dendritic cell, methods of enhancing CTL activation, and methods of inducing an immune response to cancers, bacteria, and/or viruses. The inventors also surprisingly found that introducing at least two or a plurality of the disease or target specific antigen into the ATx system increases the CTL activation.

In one aspect, the invention relates to a composition comprising (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least two repeats of a disease-specific antigen. In some embodiments, the composition further comprises a pharmaceutically-acceptable carrier or adjuvant.

In one aspect, the invention relates to a method of delivering a disease-specific antigen into a dendritic cell, the method comprising contacting the dendritic cell with a composition comprising (a) a native-receptor-ablated anthrax toxin protective antigen (PA) fused to a receptor-binding moiety specific for a target receptor on the dendritic cell and (b) a lethal factor (LF) or a fragment thereof fused to an active moiety comprising at least one repeat of the disease-specific antigen.

In one aspect, the invention relates to a method of inducing an immune response in a subject, the method comprising administering to the subject a composition comprising (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least one repeat of a disease-specific antigen. In some embodiments, the immune response is a protective immune response.

In yet another aspect, the invention relates to a method of enhancing cytotoxic-T lymphocyte (CTL) activation in a subject, the method comprising administering to the subject a composition comprising (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least one repeat of a disease-specific antigen.

In some embodiments of any one of the preceding aspects, the target receptor is selected from the group consisting of CD11c, DEC205/CD205, CD11b, CD206, CD209, Dectin-2, CD207, CD103, CD1d1, CD141/BDCA-1, CD68, CD1c/BDCA-1, and XCR1

In some embodiments of any one of the preceding aspects, the disease-specific or target antigen is selected from the group consisting of a cancer antigen, a bacterial antigen, and a viral antigen.

In some embodiments of any one of the preceding aspects, the disease-specific antigen is selected from the group consisting of: cancer antigen 125; cancer antigen 15-3; cancer antigen 19-9; prostate cancer antigen 3; alphafetoprotein; carcinoembryonic antigen; epithelial tumor antigen; tyrosinase; a human Papillomavirus 16 peptide; a human P53 peptide; a human immunodeficiency virus peptide; an MUC-I human cancer antigen peptide; a peptide from proteins of MAGE gene family; a Listeriolysin-O peptide; a P60 peptide; a MART-1 peptide; a BAGE-1 peptide; a P1A peptide; a Connexin gap junction derived peptide; a peptide or protein from one of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, and Epstein-Barr virus; and a peptide fragment of any one of the above proteins.

In some embodiments of any one of the preceding aspects, the active moiety comprises a plurality of repeats of the disease-specific antigen.

In some embodiments of any one of the preceding aspects, the plurality of the repeats of the disease-specific antigen is in the range of 2-50.

In some embodiments of any one of the preceding aspects, the plurality of the repeats of the disease-specific antigen is in the range of 2-30.

In some embodiments of any one of the preceding aspects, the plurality of the repeats of the disease-specific antigen is in the range of 3-20.

In some embodiments of any one of the preceding aspects, the plurality of the repeats of the disease-specific antigen is fused together.

In some embodiments of any one of the preceding aspects, the plurality of the repeats of the disease-specific antigen is arranged in a linear, branched, or circular manner.

In some embodiments of any one of the preceding aspects, the dendritic cell is a mammalian cell.

In some embodiments of any one of the preceding aspects, the dendritic cell is a human cell.

In some embodiments of the method of inducing an immune response, the induced immune response is against a cancer.

In some embodiments of the method of inducing an immune response, the induced immune response is against a bacterial infection.

In some embodiments of the method of inducing an immune response, the induced immune response is against a viral infection.

In some embodiments of the method of inducing an immune response, the administering is systemic.

In some embodiments of the method of inducing an immune response, the administering is performed once.

In some embodiments of the method of inducing an immune response, the administering is performed at least two times.

### BRIEF DESCRIPTION OF THE DRAWINGS

FIG. 1 is a graph demonstrating that DC-targeted ATx activates OVA-specific CTLs more efficiently than DC-targeted DT in vitro.

FIG. 2 is a graph demonstrating that delivery of OVA repeats by mAT-DTR enhances CTL response in vitro.

FIGS. 3A-3B are graphs demonstrating that DC-targeted ATx enhances activation and proliferation of CTLs in vivo.

FIG. 4 is a flow chart outlining the steps in measuring IFNγ and TNFα produced by CTLs after delivery of OVA by different toxin systems in vivo.

FIG. 5 is a graph demonstrating that delivery of OVA by DC-targeted ATx induces strong IFN≢ and TNFα production by CTLs in vivo. *p<0.05, ***p<0.001.

FIG. 6 is a flow chart outlining the steps in confirming that mAT-DTR specifically delivers OVA to DCs expressing DTR.

FIG. 7 is a graph demonstrating that DT treatment does not deplete efficiently CD11c⁺ cells.

FIG. 8 is a graph demonstrating that DT treatment decreases CD11c⁺cells and leads to a diminished CTL activation after mAT-DTR+LF_{N}-OVA immunization.

FIG. 9 is an illustration showing that modified ATx (mAT-αCD11c) binds to CD11c on the surface of DCs and transports LF_{N}-OVA to the cytosol, and LF_{N}-OVA is degraded in the cytosol by the proteosome, delivered to the ER by TAP and presented on MHC I for CTL recognition.

FIG. 10 is a flow chart outlining the steps in comparing delivery of OVA antigen in vitro by different toxin systems.

FIG. 11 is a graph demonstrating that mAT-DTR+LF_{N}-OVA activates CTLs better than DC-targeted mAT-αCD11c+LF_{N}-OVA.

FIG. 12 is a flow chart outlining the steps in determining whether delivery of OVA repeats by mAT-αCD11c enhances CTL responses.

FIG. 13 is a graph demonstrating that delivery of OVA repeats by mAT-αCD11 c does not induce robust CTL activation in vitro in that particular experiment.

FIG. 14 is a flow chart outlining the steps in determining the magnitude of CTL activation in vivo by DC-targeted mAT-αCD11c+LF_{N}-OVA.

FIG. 15 is a set of graphs demonstrating that wtAT+LF_{N}-OVA induces better CTL proliferation than DC-targeted mAT-αCD11c+LF_{N}-OVA.

FIG. 16 is a set of graphs demonstrating that wtAT+LF_{N}-OVA activates CTLs better than DC-targeted mAT-αCD11c +LF_{N}-OVA.

FIG. 17 is a set of graphs demonstrating that CTLs produce more IFNγ when OVA is delivered by wtAT as compared to delivery by DC-targeted mAT-αCD11c.

FIG. 18 is a flow chart outlining the steps in using DC-targeted ATx as a therapeutic strategy against tumors.

FIG. 19 is a flow chart outlining the steps in using DC-targeted ATx as a prophylactic strategy against tumors.

FIG. 20 is a graph demonstrating that mPA-DTR+LFN-OVA or wtPA+LFN-OVA treatments prevent tumor growth. Mice were injected with EG7-OVA cells and then treated according to the protocol shown in FIG. 18.

FIG. 21 is an image of tumors, demonstrating that mPA-DTR+LFN-OVA or wtPA+LFN-OVA treatments prevent tumor growth. Mice were injected with EG7-OVA cells and then treated according to the protocol shown in FIG. 18.

FIG. 22 is a flow chart outlining the steps in using DC-targeted ATx as a prophylactic strategy against tumors.

FIG. 23 is a graph demonstrating that immunization of mice with mPA-DTR+LFN-OVA or wtPA+LFN-OVA prevents tumor development. Mice were injected with EG7-OVA cells and treated prophylactically according to the protocol shown in FIG. 22.

FIG. 24 depicts an image of tumors and a graph demonstrating that immunization of mice with mPA-DTR+LFN-OVA or wtPA+LFN-OVA prevents tumor development. Mice were injected with EG7-OVA cells and treated prophylactically according to the protocol shown in FIG. 22.

### DETAILED DESCRIPTION

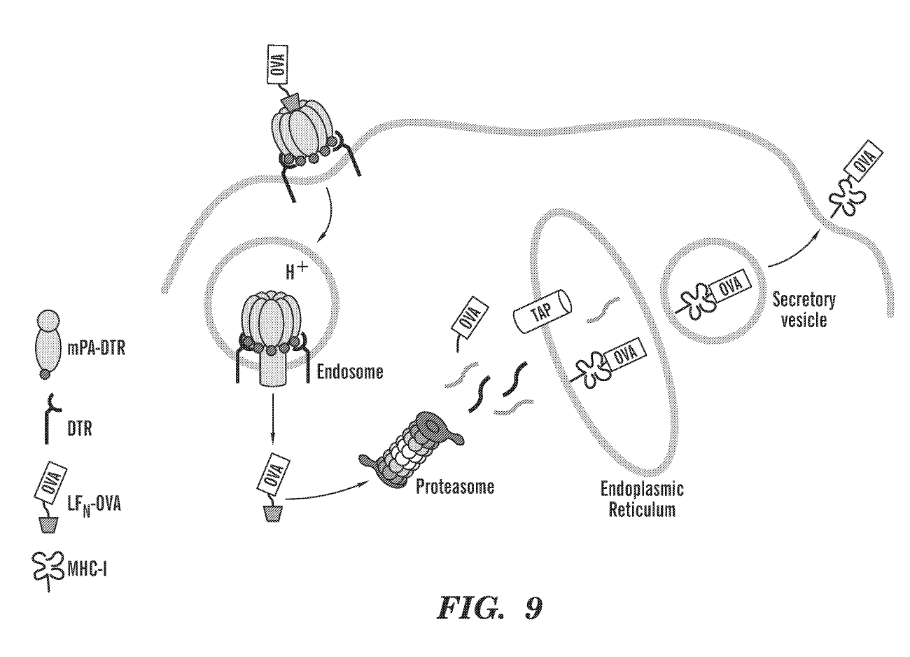

The engineered ATx systems described herein exploit the pore-forming and endocytotic capabilities of ATx. ATx is a binary toxin composed of a receptor-binding and pore-forming moiety, named Protective Antigen (PA), which is responsible for binding and actively transporting its enzymatic effectors—Lethal Factor (LF) and Edema Factor (EF)—from the extracellular milieu to the cytosol. The ATx systems described herein can be engineered by (i) ablating the native receptor on the PA to generate a PA variant, (ii) fusing the PA variant to a receptor-binding moiety specific for a target receptor on a dendritic cell, and (iii) fusing the LF to an active moiety comprising at least one repeat of a disease-specific antigen. The ATx systems can target dendritic cells in a subject and deliver a payload (e.g., a disease-specific antigen) into the cytosol of the dendritic cells. Without wishing to be bound by theory, the delivery mechanism can be see, e.g., in FIG. 9.

Various compositions and/or methods of modifying anthrax toxin for the delivery of compounds into cells have been described, e.g., in US2003/0202989 and WO2013/126690. Specifically, US2003/0202989 describes the delivery of an antigenic compound by an engineered ATx system comprising a polycationic affinity handle. WO2013/126690describes fusion molecules comprising a receptor-ablated PA fused to a non-toxin-associated receptor-binding ligand specific for a target cell. However, neither US2003/0202989 nor WO2013/126690 teaches or suggests how to target dendritic cells using the ATx systems or discloses the advantage of using multiple repeats of target or disease specific antigen in the constructs to enhance CTL response

It has been known in the art that targeted delivery of compounds into dendritic cells is challenging particularly due to the membrane modifications that occur at the time the cell encounters an antigen. The inventors surprisingly found that the engineered ATx systems described herein, when directed to receptors on DC membrane can deliver antigens such as disease-specific antigens into dendritic cells and result in enhanced CTL activation.

The engineered ATx systems described herein can comprise (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least one repeat of a disease-specific antigen.

Methods of ablating the native receptor on the anthrax toxin PA, fusing the modified PA to a receptor-binding moiety, or fusing the LF to a polypeptide or peptide can be found, e.g., in WO2013/126690, the contents of which are incorporated herein by reference. For example, the native receptor can be ablated through mutations or truncations in domain 4 of the PA.

A fragment of the LF can include a portion of the LF responsible for binding to the PA. In some embodiments, a fragment of the LF can comprise all of or a portion of the amino acids at positions 1-263 of the LF (e.g., of SEQ ID NO: 1). In some embodiments, a fragment of the LF comprises at least 5% of the amino acids at positions 1-263 of the LF (e.g., of SEQ ID NO: 1). In some embodiments, a fragment of the LF comprises at least 10% of the amino acids at positions 1-263 of the LF (e.g., of SEQ ID NO: 1). In some embodiments, a fragment of the LF comprises at least 20% of the amino acids at positions 1-263 of the LF (e.g., of SEQ ID NO: 1).

In some embodiments, a fragment of the LF comprises at least 30% of the amino acids at positions 1-263 of the LF (e.g., of SEQ ID NO: 1). In some embodiments, a fragment of the LF comprises at least 40% of the amino acids at positions 1-263 of the LF (e.g., of SEQ ID NO: 1). In some embodiments, a fragment of the LF comprises at least 50% of the amino acids at positions 1-263 of the LF (e.g., of SEQ ID NO: 1). In some embodiments, a fragment of the LF comprises at least 60% of the amino acids at positions 1-263 of the LF (e.g., of SEQ ID NO: 1). In some embodiments, a fragment of the LF comprises at least 70% of the amino acids at positions 1-263 of the LF(e.g., of SEQ ID NO: 1). In some embodiments, a fragment of the LF comprises at least 80% of the amino acids at positions 1-263 of the LF (e.g., of SEQ ID NO: 1). In some embodiments, a fragment of the LF comprises at least 90% of the amino acids at positions 1-263 of the LF (e.g., of SEQ ID NO: 1).

The amino acid sequence of the LF is shown below (SEQ ID NO: 1):

MNIKKEFIKVISMSCLVTAITLSGPVFIPLVQGAGGHGDVGMHVKEKEKN
KDENKRKDEERNKTQEEHLKEIMKHIVKIEVKGEEAVKKEAAEKLLEKVP
SDVLEMYKAIGGKIYIVDGDITKHISLEALSEDKKKIKDIYGKDALLHEH
YVYAKEGYEPVLVIQSSEDYVENTEKALNVYYEIGKILSRDILSKINQPY
QKFLDVLNTIKNASDSDGQDLLFTNQLKEHPTDFSVEFLEQNSNEVQEVF
AKAFAYYIEPQHRDVLQLYAPEAFNYMDKFNEQEINLSLEELKDQRMLAR
YEKWEKIKQHYQHWSDSLSEEGRGLLKKLQIPIEPKKDDIIHSLSQEEKE
LLKRIQIDSSDFLSTEEKEFLKKLQIDIRDSLSEEEKELLNRIQVDSSNP
LSEKEKEFLKKLKLDIQPYDINQRLQDTGGLIDSPSINLDVRKQYKRDIQ
NIDALLHQSIGSTLYNKIYLYENMNINNLTATLGADLVDSTDNTKINRGI
FNEFKKNFKYSISSNYMIVDINERPALDNERLKWRIQLSPDTRAGYLENG
KLILQRNIGLEIKDVQIIKQSEKEYIRIDAKVVPKSKIDTKIQEAQLNIN
QEWNKALGLPKYTKLITFNVHNRYASNIVESAYLILNEWKNNIQSDLIKK
VTNYLVDGNGRFVFTDITLPNIAEQYTHQDEIYEQVHSKGLYVPESRSIL
LHGPSKGVELRNDSEGFIHEFGHAVDDYAGYLLDKNQSDLVTNSKKFIDI
FKEEGSNLTSYGRTNEAEFFAEAFRLMHSTDHAERLKVQKNAPKTFQFIN
DQIKFIINS

Amino acids 1-33 (SEQ ID NO: 2) encompass the signal peptide in this sequence and amino acids 34-809 (SEQ ID NO: 3) encompass the Lethal Factor protein.

PA binding capacity of LF has been studied using mutagenesis. The compositions and methods of the present invention include variants that do not abolish the capacity of the LF to bind PA. Non-limiting examples of such variants include can be seen in Table 1.

TABLE 1
Mutations in LF that have no effect on PA-binding ability

| Position | Mutation |
|---|---|
| 180 | V to A |
| 183 | E to A |
| 185 | G to A |
| 221 | L to A |
| 222 | L to A |

In some embodiments, the epitope or antigen comprises at least 4 consecutive amino acids. In some embodiments, the epitope or antigen comprises at least 5 consecutive amino acids. In some embodiments, the epitope or antigen comprises at least 6 consecutive amino acids. In some embodiments, the epitope or antigen comprises at least 7 consecutive amino acids. In some embodiments, the epitope or antigen comprises at least 8 consecutive amino acids. In some embodiments, the epitope or antigen comprises at least 9 consecutive amino acids. In some embodiments, the epitope or antigen comprises at least 10 consecutive amino acids. In some embodiments, the epitope or antigen comprises no more than 25 consecutive amino acids. In some embodiments, the epitope or antigen comprises no more than 20 consecutive amino acids. In some embodiments, the epitope or antigen comprises 4-25 consecutive amino acids. In some embodiments, the epitope or antigen comprises 4-20 consecutive amino acids. In some embodiments, the epitope or antigen comprises 4-15 consecutive amino acids. In some embodiments, the epitope or antigen comprises 10-25 consecutive amino acids.

The epitope or antigen can have a variety of conformations such as linear, circular, or 3-dimensional.

In some embodiments, the active moiety comprises a plurality of repeats of the disease-specific antigen (e.g., 2, 3, 4, 5, or more). The number of repeats should not be so high that the active moiety cannot translocate through the cell membrane. But based on our experience using ATx systems, and depending on the size of the antigen, one can add at least 5000 repeats, likely up to at least 10,000 repeats. The plurality of repeats of the disease-specific antigen permits repetitive delivery of the same antigen to the dendritic cells. In some aspects of all the embodiments, the active moiety comprises up to 10,000 repeats of the disease-specific antigen. In some aspects of all the embodiments, the active moiety comprises up to 5,000 repeats of the disease-specific antigen. In some aspects of all the embodiments, the active moiety comprises up to 1,000 repeats of the disease-specific antigen. In some aspects of all the embodiments, the active moiety comprises up to 500 repeats of the disease-specific antigen. In some aspects of all the embodiments, the active moiety comprises up to 250 repeats of the disease-specific antigen. In some aspects of all the embodiments, the active moiety comprises 2-500 repeats of the disease-specific antigen. In some aspects of all the embodiments, the active moiety comprises 2-400 repeats of the disease-specific antigen. In some aspects of all the embodiments, the active moiety comprises 2-300 repeats of the disease-specific antigen. In some aspects of all the embodiments, the active moiety comprises 2-200 repeats of the disease-specific antigen. In some aspects of all the embodiments, the active moiety comprises 2-100 repeats of the disease-specific antigen. In some aspects of all the embodiments, the active moiety comprises 2-50 repeats of the disease-specific antigen.

In some embodiments, the plurality of the repeats of the disease-specific antigen is fused together. The plurality of the repeats of the disease-specific antigen can be arranged in a variety of manners such as, but not limited to, a linear chain, a branched structure, a circular structure, or a combination thereof.

The target receptor is on the surface of the dendritic cells. In some aspects of all the embodiments, the target receptor is specific to the dendritic cells. The term “specific” means that the receptor is only found on dendritic cells and not present on other cells in measurable amounts. In some aspects of all the embodiments, the target receptor can also be present on other cell types. In some aspects of all the embodiments, the target receptor is selected based on it being present only in only 1-5, 1-4, 1-3, 1-2 or 1 other different cell types to limit the targeting mostly to DC. In some aspects of all the embodiments, the target receptor is selected from the group consisting of CD11c, DEC205/CD205, CD11b, CD206, CD209, Dectin-2, CD207, CD103, CD1d1, CD1D, CD141/BDCA-1, CD68, CD1c/BDCA-1, and XCR1.

In some aspects of all the embodiments, the target is selected from receptors that are known to continue to be present on the cells during the membrane reorganization when DC encounters an antigen. In some aspects of all the embodiments, the target is selected from receptors that are not downregulated on the cells during the membrane reorganization. In some aspects of all the embodiments of the invention, the receptor is selected from XCR1 and DEC205/CD205.

CD11c, also known as Integrin, alpha X (complement component 3 receptor 4 subunit) (ITGAX), is a gene that encodes for CD11c. CD11c is an integrin alpha X chain protein. Integrins are heterodimeric integral membrane proteins composed of an alpha chain and a beta chain. This protein combines with the beta 2 chain (ITGB2) to form a leukocyte-specific integrin referred to as inactivated-C3b (iC3b) receptor 4 (CR4). The alpha X beta 2 complex seems to overlap the properties of the alpha M beta 2 integrin in the adherence of neutrophils and monocytes to stimulated endothelium cells, and in the phagocytosis of complement coated particles. CD11c is a type I transmembrane protein found at high levels on most human dendritic cells, but also on monocytes, macrophages, neutrophils, and some B cells that induces cellular activation and helps trigger neutrophil respiratory burst; expressed in hairy cell leukemias, acute nonlymphocytic leukemias, and some B-cell chronic lymphocytic leukemias.

CD205 is an endocytic receptor that is expressed at high levels by cortical thymic epithelial cells and by dendritic cell (DC) subsets, including the splenic CD8+ DC population that is responsible for cross-presentation of apoptotic cell-derived antigens.

Cluster of differentiation molecule 11B (CD11B), also known as integrin alpha M (ITGAM) is one protein subunit that forms the heterodimeric integrin alpha-M beta-2 (αMβ2) molecule, also known as macrophage-1 antigen (Mac-1) or complement receptor 3 (CR3). ITGAM is also known as CR3A. The second chain of αMβ2 is the common integrin β2 subunit known as CD18, and integrin αMβ2 thus belongs to the β2 subfamily (or leukocyte) integrins. αMβ2 is expressed on the surface of many leukocytes involved in the innate immune system, including monocytes, granulocytes, macrophages, and natural killer cells. It mediates inflammation by regulating leukocyte adhesion and migration and has been implicated in several immune processes such as phagocytosis, cell-mediated cytotoxicity, chemotaxis and cellular activation. It is involved in the complement system due to its capacity to bind inactivated complement component 3b (iC3b). The ITGAM (alpha) subunit of integrin αMβ2 is directly involved in causing the adhesion and spreading of cells but cannot mediate cellular migration without the presence of the β2 (CD18) subunit.

CD206 is widely known as mannose receptor C type 1 (MRC1) which is part of the mannose receptor (MR) family. All members of this family share a common extracellular domain structure, but with distinct ligand binding properties and cell type expression. This is a 162-175 kDa type -1 transmembrane protein and a member of the Group VI C-type lectins along with CD280 (ENDO180), CD205 (DEC205), and the phospholipase A2 receptor (PLA2R1). CD206 is a complex molecule composed of a N-terminal cysteine-rich ricin b-type lectin domain (RICIN), a fibronectin type II domain (FN2), eight tandemly arranged C-type lectin like domains (CTLDs), a transmembrane domain (TM), and a cytoplasmic domain. The terminal cysteine-rich domain of CD206 binds sulphated sugars, while CTLDs 4 to 8 recognize polysaccharides terminated in mannose, fucose, or N-acetylglucosamine. These sugars are all found on microorganisms and on some endogenous glycoproteins. CD206 is found on numerous cell types, including: tissue macrophages, lymphatic and hepatic epithelium, kidney mesangial cells, tracheal smooth muscle, retinal pigment epithelium, human monocyte derived dendritic cells, and some subpopulations of mouse dendritic cells. CD206 is also active in endocytosis and phagocytosis.

CD209, known as Dendritic Cell-Specific Intercellular adhesion molecule 3 (ICAM-3)-Grabbing Nonintegrin (DC-SIGN), is a 44 kD type II transmembrane glycoprotein and a member of the C-type lectin family. CD209 is expressed on myeloid dendritic cells, placental macrophages, liver and placental endothelial cells.

Dectin-2 is a type II transmembrane CLR that was originally cloned from a DC line (Ariizumi, K., et al. 2000. J. Biol. Chem. 275:11957-11963) but is most abundantly expressed on tissue macrophages and inflammatory monocytes and has specificity for high mannose structures (Taylor, P. R. et al., 2005. Eur. J. Immunol. 35:2163-2174; McGreal, E. P., et al.,2006. Glycobiology. 16:422-430).

Langerin (CD207) is a cell surface C-type lectin located on Langerhans cells (LCs), specialized skin dendritic cells (DCs) that take up and degrade antigens for presentation to the immune system. Langerin can be internalized and accumulates in Birbeck granules (BGs), subdomains of the endosomal recycling compartment that are specific to Langerhans cells. Langerin binds and mediates uptake and degradation of glycoconjugates containing mannose and related sugars, and these properties may allow langerin to play a role in antigen uptake and processing (Ward, E. M., et al., J. Biol. Chem. 281: 15450-15456, 2006).

CD103 (cluster of differentiation 103), also known as integrin, alpha E (ITGAE) is an integrin protein that in human is encoded by the ITGAE gene. CD103 binds integrin beta 7 (β7-ITGB7) to form the complete heterodimeric integrin molecule αEβ7, which has no distinct name. The αEβ7 complex is often referred to as “CD103” though this appellation strictly refers only to the αE chain. CD103 is expressed widely on intraepithelial lymphocyte (IEL) T cells (both αβT cells and γδ T cells) and on some peripheral regulatory T cells (Tregs). It has also been reported on lamina propria T cells. [4] A subset of dendritic cells in the gut mucosa and in mesenteric lymph nodes also expresses this marker and is known as CD103 DCs.

Human CD141 (BDCA-3) antigen which is expressed at high levels on a minor subpopulation of human myeloid dendritic cells (about 0.02% of blood leukocytes). CD141 is also known as thrombomodulin; thrombomodulin mediates co-agglutination by interaction with thrombin and protein C.

CD68 has been identified on epidermal dendritic cells (Petzelbauer et al. J Invest Dermatol. 1993 September; 101(3):256-61). It is detected primarily on monocytes and macrophages and is considered a pan-macrophage antigen). Other cell types that have been found to express CD68 are astrocytes, basophils, B-cells, CD34(+) progenitor cells (Strobl et al, 1995), chondrocytes, dendritic cells and their precursors, epithelial cells (Travaglione et al, 2002), fibroblasts, foam cells, Hofbauer cells, hyalocytes, Kupffer cells, Langerhans cells, macrophages, mast cells, melanoma cells, microglial cells, monocytes, neutrophils, NK-cells, osteoblast-like cells (Heinemann et al, 2000), osteoclasts, platelets after cell activation, podocytes, Reed-Sternberg cells, retinal pigment epithelial cells (Einer et al, 1992), Schwann cells, synoviocytes, T-cells. CD68 is a heavily O-glycosylated mucin-like membrane protein with significant sequence homology of the membrane proximal and cytoplasmic domains to a family of lysosomal/plasma membrane shuttling proteins (represented, e. g., by LAMP-1) (Holness and Simmons, 1993; Holness et al, 1993).

CD1c/BDCA-1 encodes a member of the CD1 family of transmembrane glycoproteins, which are structurally related to the major histocompatibility complex (MHC) proteins and form heterodimers with beta-2-microglobulin. The CD1 proteins mediate the presentation of primarily lipid and glycolipid antigens of self or microbial origin to T cells.

XCR1 is also known as GPR5. The protein encoded by this gene is a chemokine receptor belonging to the G protein-coupled receptor superfamily. The family members are characterized by the presence of 7 transmembrane domains and numerous conserved amino acids. This receptor is most closely related to RBS11 and the MIP1-alpha/RANTES receptor. It transduces a signal by increasing the intracellular calcium ions level. The viral macrophage inflammatory protein-II is an antagonist of this receptor and blocks signaling.

In some aspects of all the embodiments, the disease specific antigen can be a cancer or tumor antigen or a fragment thereof. A number of cancer antigens are known and the compositions and methods are not intended to be limited to any particular cancer antigen. The compositions and methods of the present invention allow use of any or a combination of two or more cancer antigens. Thus, any and all of the cancer antigens, or any suitable combination of two or more of the antigens are contemplated as suitable antigens for the compositions and methods set forth in the present invention.

In some aspects of all the embodiments, one can use a plurality of two different cancer antigens in one ATx delivery system to allow induction and/or enhancement of CTL response to two different cancer antigens.

Tumor or cancer antigen is an antigenic substance produced in tumor cells, i.e., it triggers an immune response in the host. Tumor antigens are useful tumor markers in identifying tumor cells with diagnostic tests and are potential candidates for use in cancer therapy.

Currently, tumor antigens are often divided into: Products of Mutated Oncogenes and Tumor Suppressor Genes; Products of Other Mutated Genes Overexpressed or Aberrantly Expressed Cellular Proteins; Tumor Antigens Produced by Oncogenic Viruses; Oncofetal Antigens; Altered Cell Surface Glycolipids and Glycoproteins; Cell Type-Specific Differentiation Antigens.

Practically any protein produced in a tumor cell that has an abnormal structure due to mutation can act as a tumor antigen. Such abnormal proteins are produced due to mutation of the concerned gene. Mutation of protooncogenes and tumor suppressors which lead to abnormal protein production are the cause of the tumor and thus such abnormal proteins are called tumor-specific antigens. Examples of tumor-specific antigens include the abnormal products of ras and p53 genes. In contrast, mutation of other genes unrelated to the tumor formation may lead to synthesis of abnormal proteins which are called tumor-associated antigens.

Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be much more specific to cancer cells because normal cells shouldn't contain these proteins. Normal cells will display the normal protein antigen on their MHC molecules, whereas cancer cells will display the mutant version. Some viral proteins are implicated in forming cancer (oncogenesis), and some viral antigens are also cancer antigens. Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens. Example antigens of this type are CTAG1B and MAGEA1.

Proteins that are normally produced in very low quantities but whose production is dramatically increased in tumor cells, trigger an immune response. An example of such a protein is the enzyme tyrosinase, which is required for melanin production. Normally tyrosinase is produced in minute quantities but its levels are very much elevated in melanoma cells.

Oncofetal antigens are another important class of tumor antigens. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens.

Abnormal proteins are also produced by cells infected with oncoviruses, e.g. EBV and HPV. Cells infected by these viruses contain latent viral DNA which is transcribed and the resulting protein produces an immune response.

In addition to proteins, other substances like cell surface glycolipids and glycoproteins may also have an abnormal structure in tumor cells and could thus be targets of the immune system.

Other examples of known cancer antigens include, but are not limited to, cancer antigen 125, cancer antigen 15-3, cancer antigen 19-9, prostate cancer antigen 3, alphafetoprotein, carcinoembryonic antigen, epithelial tumor antigen, tyrosinase, MUC-I human cancer antigen, Melanoma-associated antigen, MART-1, B melanoma antigen, P1A, and P53.

In some embodiments, the disease specific antigen can be an antigen or a fragment thereof associated with a pathogen such as a bacterium or virus. Examples of pathogens include, but are not limited to, Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, Epstein-Barr virus, Ebola, human immunodeficiency virus, human Papillomavirus, and Listeria monocytogenes.

Some non-limiting examples of disease specific antigens are as follows: Human Papillomavirus 16 peptides (e.g., antigens E6 and E7, E7 peptide 49-57 RAHYNIVTF); human P53 peptides (e.g., V10 peptide FYQLAKTCPV); human immunodeficiency virus peptides (e.g., gp 120, P18 peptide RIQRGPGRAFVTIGK); MUC-I human cancer antigen peptides; peptides from proteins of MAGE gene family (e.g., MAGE-1 SAYGEPRKL, MAGE-3 FLWGPRALV); peptides from the human tyrosinase protein (e.g., Tyr-A2-1 MLLAVLYCL, Try-A@-2 YMNGTMSQV); Listeriolysin-O peptides e.g., LLO₉₁₋₉₉GYKDGNEYI); P60 peptides (e.g., P60217-225 KYGVSVQDI); MART-1 peptides (e.g., M-9 AAAAAGIGILTV, M10-3 EAAGIGILTV); BAGE-1 peptides (e.g., AARAVFLAL); P1A peptides (e.g., P815A35-43 LPYLGWLVF); Connexin gap junction derived peptides (e.g., Mut 1 FEQNTAQP, MUT 2 FEQNTAQA); peptides/proteins from any of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I,II, Varicella zoster, Variola, Polio, Yellow Fever, Encephalitis viruses, and Epstein-Barr virus.

In some embodiments, the active moiety can comprise at least two types of disease-specific antigen (e.g., 2, 3, 4, 5, or more different antigens). For example, the active moiety can comprise a first cancer antigen and a second cancer antigen, each of which can comprise repeats. Alternatively, the active moiety can comprise a cancer antigen and a bacterial antigen, each of which can comprise repeats. The active moiety can also comprise a cancer antigen and a viral antigen, each of which can comprise repeats. The active moiety can also comprise a cancer antigen, a viral antigen, and a bacterial antigen, each of which can comprise repeats. This arrangement can permit the delivery of different types of disease-specific antigen to induce an immune response against two or more infections or cancers in a single dose.

In some aspects of all the embodiments, the compositions comprising the engineered ATx systems described herein can further comprise one or more adjuvants. As used herein, an “adjuvant” is a substance that serves to enhance the immunogenicity of a composition that can induce an immune response. Thus, adjuvants are often given to boost the immune response and are well known to the skilled artisan. Suitable adjuvants to enhance effectiveness of the composition include, but are not limited to:

(1) aluminum salts, such as aluminum hydroxide, aluminum phosphate, aluminum sulfate, hydrated alumina, alumina hydrate, alumina trihydrate (ATH), aluminum hydrate, aluminum trihydrate, alhydrogel, Superfos, Amphogel, aluminum (III) hydroxide, aluminum hydroxyphosphate sulfate (Aluminum Phosphate Adjuvant (APA)), amorphous alumina, trihydrated alumina, or trihydroxyaluminum.etc.;

(2) oil-in-water emulsion formulations (with or without other specific immunostimulating agents such as muramyl peptides or bacterial cell wall components), such as, for example, (a) MF59 (PCT Publ. No. WO 90/14837), containing 5% Squalene, 0.5% Tween 80, and 0.5% Span 85 (optionally containing various amounts of MTP-PE (see below, although not required)) formulated into submicron particles using a microfluidizer such as Model 110Y microfluidizer (Microfluidics, Newton, Mass.); (b) SAF, containing 10% Squalene, 0.4% Tween 80, 5% pluronic-blocked polymer L121, and thr-MDP (see below) either microfluidized into a submicron emulsion or vortexed to generate a larger particle size emulsion; and (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094 (Corixa), trehalose dimycolate (TDM), and cell wall skeleton (CWS), preferably MPL+CWS (Detox™);

(3) saponin adjuvants, such as Quil A or STIMULON™ QS-21 (Antigenics, Framingham, Mass.) (U.S. Pat. No. 5,057,540) may be used or particles generated therefrom such as ISCOMs (immunostimulating complexes);

(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-Tetradecanoyloxytetradecanoylamino]ethyl 2-Deoxy-4-O-phosphono-3-O—[(R)-3-tetradecanoyloxytetradecanoyl]-2-[(R)-3-tetradecanoyloxytetradecanoylamino]-b-D-glucopyranoside, which is also known as 529 (formerly known as RC529), which is formulated as an aqueous form or as a stable emulsion, synthetic polynucleotides such as oligonucleotides containing CpG motif(s) (U.S. Pat. No. 6,207,646);

(5) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.;

(6) detoxified mutants of a bacterial ADP-ribosylating toxin such as a cholera toxin (CT) either in a wild-type or mutant form, for example, where the glutamic acid at amino acid position 29 is replaced by another amino acid, preferably a histidine, in accordance with published international patent application number WO 00/18434 (see also WO 02/098368 and WO 02/098369), a pertussis toxin (PT), or an E. coli heat-labile toxin (LT), particularly LT-K63, LT-R72, CT-S109, PT-K9/G129 (see, e.g., WO 93/13302 and WO 92/19265); and

(7) other substances that act as immunostimulating agents to enhance the effectiveness of the composition.

Muramyl peptides include, but are not limited to, N-acetyl-muramyl-L-threonyl-D-isoglutamine (thr-MDP), N-acetyl-normuramyl-L-alanine-2-(1′-2′dipalmitoyl-sn-glycero-3-hydroxyphosphoryloxy)-ethylamine (MTP-PE), etc.

In some embodiments, the composition further comprises a pharmaceutically-acceptable carrier. As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the composition further comprises one or more pharmaceutically-acceptable diluents, buffers, stabilizers, preservatives, and/or emulsifiers. Examples of preservatives include, but are not limited to, 2-phenoxyethanol and thiomersal. Examples of stabilizers include, but are not limited to, sucrose, mannitol, lactose, and gelatin. Examples of emulsifiers include, but are not limited to, polysorbate-80 and sorbitol.

In some embodiments, the compositions described herein can induce a protective immune response sufficient as vaccines against a cancer. In some embodiments, the compositions described herein can induce a protective immune response sufficient as vaccines against a bacterial infection. In some embodiments, the compositions described herein can induce a protective immune response sufficient as vaccines against a viral infection.

An immune response is generated, in general, as follows: T cells recognize proteins when the protein has been cleaved into smaller peptides and is presented in a complex called the “major histocompatability complex (WIC)” located on another cell's surface. There are two classes of MEW complexes—class I and class II, and each class is made up of many different alleles. Different patients can have different types of WIC complex alleles.

Reference to “protective” immunity or immune response, when used in the context of a polypeptide, immunogen and/or treatment method described herein, indicates a detectable level of protection against a cancer or an infection. This includes therapeutic and/or prophylactic measures reducing the likelihood of a cancer or an infection or of obtaining a disorder(s) resulting from such infection, as well as reducing the severity of the infection and/or a disorder(s) resulting from such infection. As such, a protective immune response includes, for example, the ability to reduce bacterial or viral load, ameliorate one or more disorders or symptoms associated with said bacterial or viral infection, and/or delaying the onset of disease progression resulting from such infection. The level of protection can be assessed using animal models. A protective immune response can be measured, for example, by flow cytometry, development of antibodies, or by measuring resistance to pathogen challenge in vivo. A protective immune response can also be determined by charactering the memory T cell pool after immunization.

An effective amount of the compositions comprising the engineered ATx systems described herein can be administered for delivering a disease-specific antigen into a dendritic cell, inducing an immune response in a subject, or enhancing cytotoxic-T lymphocyte (CTL) activation in a subject. The effective amount can be determined experimentally without undue experimentation using routine methods to detect a CTL response.

CTL activation can be determined by methods known in the art, e.g., by measuring the level of IFNγ and TNFα after the administration of the engineered ATx systems described herein.

The effective amount of the engineered ATx composition to induce a protective immune response in a subject can be determined by methods involving observation of appropriate immune responses in subjects. The effective amount can be extrapolated from, for example, animal studies. This quantity can be subject-dependent, and can be determined based upon the characteristics of the subject (e.g., age, gender, race, ethnicity, or health status) and the level of immunity required. The effective amount should not induce significant adverse effects but even if it does, many instances side effects can be effectively managed using additional therapies, such as steroids.

It is not intended that the administration be limited to a particular mode of administration, dosage, or frequency of dosing. An effective amount, e.g., an immunologically effective dose of the compositions disclosed herein may be administered to the subject in a single dose or in multiple doses. When multiple doses are administered to the subject, a second or third dose can be administered days (e.g., 1, 2, 3, 4, 5, 6, 7, 8, 9, 10), weeks (e.g., 1, 2, 3, 4, 5, 6, 7, 8, 9, 10), months (e.g., 1, 2, 3, 4, 5, 6, 7, 8, 9, 10) or years (e.g., 1, 2, 3, 4, 5, 6, 7, 8, 9, 10) after the initial dose. For example, a second dose of the composition can be administered about 7 days, about 14 days, about 28 days, or within a year, following administration of a first dose of the composition.

In some aspects, the delivery is systemic. In some aspects the delivery can be local, e.g., close to a localized tumor. The delivery can be directly into the tumor or to the surrounding tissues, e.g., intramuscularly or subcutaneuously. The systemic delivery can be achieved, e.g., intravenously, intraperitoneally or orally. The delivery can also be intracranial.

In one embodiment, a composition of the present invention is administered as a single inoculation. In another embodiment, the composition is administered twice, three times, or four times or more, adequately spaced apart. For example, the composition can be administered at 1, 2, 3, 4, 5, or 6 month intervals or any combination thereof. The immunization schedule can follow that designated for the particular cancer or infection. In one embodiment, one or more booster doses can be administered at distant times as needed.

The dose can vary depending on factors such as gender, age, weight, condition of the particular subject, and the particular disease-specific antigen in the composition. In some embodiments, each dose can comprise the disease-specific antigen in the range of 0.1 μg to 1 mg.

A subject can be treated prophylactically or therapeutically. Prophylactic treatment provides sufficient protective immunity to reduce the likelihood, or severity, of a particular cancer, a bacterial infection, or a viral infection. Therapeutic treatment can be performed to reduce the severity of a cancer, a bacterial infection, or a viral infection after the cancer, bacterial infection or viral infection has been detected. The compositions of the present invention can be provided either prior to the onset of the cancer or the infection or after the initiation of an actual infection. For example, prophylactic therapy can be directed to subjects with significant family history of cancer or with particular cancer-associated germline mutations, such as BRCA1 or BRCA2 carriers. Similarly, subjects at risk of particular bacterial or viral exposure can be treated prophylactically.

The inventors have also surprisingly found that the present ATx mediated system provides significant effects already after one exposure to the treatment. As opposed to a typical immunization, which requires initial exposure and one or more booster dosages, the ATx system as described herein can provide a robust CTL activation and immune protection after only one injection. Thus, in some aspects of all the embodiments of the invention, only one administration of the ATx system is performed.

In some embodiments, an LF fused to a first type of disease-specific antigen and an LF fused to a second type of disease-specific antigen can be administered together or sequentially to allow for immunization against two or more infections/cancers. More than two types of disease-specific antigen can be targeted to dendritic cells in a similar manner.

It should be noted that the technology described herein is not limited to dendritic cells. The compositions and methods described herein can be applied to other antigen-presenting cells such as macrophages, certain B-cells, and certain activated epithelial cells.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Although any known methods, devices, and materials may be used in the practice or testing of the invention, the methods, devices, and materials in this regard are described herein.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the term “comprising” or “comprises” is used in reference to compositions, methods, and respective component(s) thereof, that are useful to an embodiment, yet open to the inclusion of unspecified elements, whether useful or not.

As used herein, the term “consisting essentially of” refers to those elements required for a given embodiment. The term permits the presence of elements that do not materially affect the basic and novel or functional characteristic(s) of that embodiment of the invention.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models for immunization. A subject can be male or female of any age, including infants, children, teenagers, and adults.

As described herein, an “antigen” is a molecule that is bound by a binding site comprising the complementarity determining regions (CDRs) of an antibody agent. Typically, antigens are bound by antibody ligands and are capable of raising an antibody response in vivo. An antigen can be a polypeptide, protein, nucleic acid or other molecule or portion thereof.

The terms “polypeptide” and “protein” are used interchangeably to refer to a polymer of amino acid residues, and are not limited to a minimum length. For example, the polymer of amino acids can comprise at least 2 amino acids (e.g., at least 5, at least 10, at least 20, at least 30, at least 40, at least 50, at least 60, at least 70, at least 80, at least 90, at least 100, at least 125, at least 150, at least 175, at least 200, at least 225, at least 250, at least 275, at least 300, at least 350, at least 400, at least 450, at least 500, at least 600, at least 700, at least 800, at least 900, at least 1000, at least 2000, at least 3000, at least 4000, at least 5000, at least 6000, at least 7000, at least 8000, at least 9000, at least 10,000 amino acids or more). Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational and post-translational modifications of the polypeptide, such as, for example, disulfide-bond formation, glycosylation, acetylation, phosphorylation, proteolytic cleavage (e.g., cleavage by furins or metalloproteases and prohormone convertases (PCs)), and the like. Furthermore, for purposes of the present invention, a “polypeptide” encompasses a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, has a well-defined conformation. Proteins, as opposed to peptides, generally consist of chains of 50 or more amino acids. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 50 amino acids in length.

The term “fragment” of a peptide, polypeptide or molecule as used herein refers to any contiguous polypeptide subset of the molecule. Accordingly, a “fragment” of a molecule, is meant to refer to any polypeptide subset of the molecule.

As used herein, the term “immune response” refers to a response of a cell of the immune system, such as a B cell, T cell, or monocyte, to a stimulus, such as a pathogen or antigen (e.g., formulated as an immunogenic composition or vaccine). An immune response can be a B cell response, which results in the production of specific antibodies, such as antigen specific neutralizing antibodies. An immune response can also be a T cell response, such as a CD4+response or a CD8+response. B cell and T cell responses are aspects of a “cellular” immune response. An immune response can also be a “humoral” immune response, which is mediated by antibodies, which can be detected and/or measured, e.g., by an ELISA assay.

As used herein, a “protective immune response” is an immune response that inhibits a detrimental function or activity of a pathogen or a cancer, reduces infection by a pathogen, decreases one or more symptoms (including death) that result from the cancer or the infection by the pathogen, and/or delaying the onset of disease progression resulting from the cancer or the infection by the pathogen.

As used herein, the term “cancer” refers to an uncontrolled growth of cells which interferes with the normal functioning of the bodily organs and systems. A subject who has a cancer is a subject having objectively measurable cancer cells present in the subject's body. Included in this definition are benign and malignant cancers, premalignant lesions, as well as dormant tumors or micrometastases. Cancers which migrate from their original location and seed vital organs can eventually lead to the death of the subject through the functional deterioration of the affected organs.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a composition as disclosed herein into a subject by a method or route which results in at least partial delivery of the agent at a desired site. Pharmaceutical compositions disclosed herein can be administered by any appropriate route which results in an effective treatment in the subject.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intrahepatic, intraperitoneal, transtracheal, subcutaneous, subcuticular, intraarticular, sub capsular, subarachnoid, intraspinal, intracerebro spinal, and intrasternal injection and infusion. The administration can be systemic or local.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean ±1% of the value being referred to. For example, about 100 means from 99 to 101.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A method of delivering a disease-specific antigen into a dendritic cell, the method comprising contacting the dendritic cell with a composition comprising (a) a native-receptor-ablated anthrax toxin protective antigen (PA) fused to a receptor-binding moiety specific for a target receptor on the dendritic cell and (b) a lethal factor (LF) or a fragment thereof fused to an active moiety comprising at least one repeat of the disease-specific antigen.
2. The method of paragraph 1, wherein the target receptor is selected from the group consisting of CD11c, DEC205/CD205, CD11b, CD206, CD209, Dectin-2, CD207, CD103, CD1d1, CD141/BDCA-1, CD68, CD1c/BDCA-1, and XCR1.
3. The method of paragraph 1 or 2, wherein the disease-specific antigen is selected from the group consisting of a cancer antigen, a bacterial antigen, and a viral antigen.
4. The method of any one of the paragraphs 1-3, wherein the disease-specific antigen is selected from the group consisting of: cancer antigen 125; cancer antigen 15-3; cancer antigen 19-9; prostate cancer antigen 3; alphafetoprotein; carcinoembryonic antigen; epithelial tumor antigen; tyrosinase; a human Papillomavirus 16 peptide; a human P53 peptide; a human immunodeficiency virus peptide; an MUC-I human cancer antigen peptide; a peptide from proteins of MAGE gene family; a peptide from human tyrosinase protein; a Listeriolysin-O peptide; a P60 peptide; a MART-1 peptide; a BAGE-1 peptide; a P1A peptide; a Connexin gap junction derived peptide; a peptide or protein from one of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, and Epstein-Barr virus.
5. The method of any one of the paragraphs 1-4, wherein the active moiety comprises a plurality of repeats of the disease-specific antigen.
6. The method of paragraph 5, wherein the plurality of the repeats of the disease-specific antigen is in the range of 2-50.
7. The method of paragraph 5, wherein the plurality of the repeats of the disease-specific antigen is in the range of 2-30.
8. The method of paragraph 5, wherein the plurality of the repeats of the disease-specific antigen is in the range of 3-20.
9. The method of any one of the paragraphs 5-8, wherein the plurality of the repeats of the disease-specific antigen is fused together.
10. The method of any one of the paragraphs 5-9, wherein the plurality of the repeats of the disease-specific antigen is arranged in a linear, branched, or circular manner.
11. The method of any one of the paragraphs 1-10, wherein the dendritic cell is a mammalian cell.
12. The method of paragraph 11, wherein the dendritic cell is a human cell.
13. The method of any one of the paragraphs 1-12, wherein the contacting is performed in vitro.
14. The method of any one of the paragraphs 1-12, wherein the contacting is performed in vivo.
15. A method of inducing an immune response in a subject, the method comprising administering to the subject a composition comprising (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least one repeat of a disease-specific antigen.
16. The method of paragraph 15, wherein the immune response is a protective immune response.
17. The method of paragraph 15 or 16, wherein the target receptor is selected from the group consisting of CD11c, DEC205/CD205, CD11b, CD206, CD209, Dectin-2, CD207, CD103, CD1d1, CD141/BDCA-1, CD68, CD1c/BDCA-1, and XCR1.
18. The method of any one of the paragraphs 15-17, wherein the disease-specific antigen is selected from the group consisting of a cancer antigen, a bacterial antigen, and a viral antigen.
19. The method of paragraph 18, wherein the induced immune response is against a cancer.
20. The method of paragraph 18, wherein the induced immune response is against a bacterial infection.
21. The method of paragraph 18, wherein the induced immune response is against a viral infection.
22. The method of any one of the paragraphs 15-21, wherein the disease-specific antigen is selected from the group consisting of: cancer antigen 125; cancer antigen 15-3; cancer antigen 19-9; prostate cancer antigen 3; alphafetoprotein; carcinoembryonic antigen; epithelial tumor antigen; tyrosinase; a human Papillomavirus 16 peptide; a human P53 peptide; a human immunodeficiency virus peptide; an MUC-I human cancer antigen peptide; a peptide from proteins of MAGE gene family; a peptide from human tyrosinase protein; a Listeriolysin-O peptide; a P60 peptide; a MART-1 peptide; a BAGE-1 peptide; a PlA peptide; a Connexin gap junction derived peptide; a peptide or protein from one of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, and Epstein-Barr virus.
23. The method of any one of paragraphs 15-22, wherein the active moiety comprises a plurality of repeats of the disease-specific antigen.
24. The method of paragraph 23, wherein the plurality of the repeats of the disease-specific antigen is in the range of 2-50.
25. The method of paragraph 23, wherein the plurality of the repeats of the disease-specific antigen is in the range of 2-30.
26. The method of paragraph 23, wherein the plurality of the repeats of the disease-specific antigen is in the range of 3-20.
27. The method of any one of the paragraphs 23-26, wherein the plurality of the repeats of the disease-specific antigen is fused together.
28. The method of any one of the paragraphs 23-27, wherein the plurality of the repeats of the disease-specific antigen is arranged in a linear, branched, or circular manner.
29. The method of any one of the paragraphs 15-28, wherein the active moiety comprises at least two types of disease-specific antigen.
30. The method of any one of the paragraphs 15-29, wherein the subject is a mammal.
31. The method of paragraph 30, wherein the mammal is a human.
32. The method of any one of the paragraphs 15-31, wherein the administering is systemic.
33. The method of any one of the paragraphs 15-32, wherein the administering is performed once.
34. The method of any one of the paragraphs 15-32, wherein the administering is performed at least two times.
35. A method of enhancing cytotoxic-T lymphocyte (CTL) activation in a subject, the method comprising administering to the subject a composition comprising (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least one repeat of a disease-specific antigen.
36. The method of paragraph 35, wherein the target receptor is selected from the group consisting of CD11c, DEC205/CD205, CD11b, CD206, CD209, Dectin-2, CD207, CD103, CD1d1, CD141/BDCA-1, CD68, CD1c/BDCA-1, and XCR1.
37. The method of paragraph 35 or 36, wherein the disease-specific antigen is selected from the group consisting of a cancer antigen, a bacterial antigen, and a viral antigen.
38. The method of any one of the paragraphs 35-37, wherein the disease-specific antigen is selected from the group consisting of: cancer antigen 125; cancer antigen 15-3; cancer antigen 19-9; prostate cancer antigen 3; alphafetoprotein; carcinoembryonic antigen; epithelial tumor antigen; tyrosinase; a human Papillomavirus 16 peptide; a human P53 peptide; a human immunodeficiency virus peptide; an MUC-I human cancer antigen peptide; a peptide from proteins of MAGE gene family; a peptide from human tyrosinase protein; a Listeriolysin-O peptide; a P60 peptide; a MART-1 peptide; a BAGE-1 peptide; a P1A peptide; a Connexin gap junction derived peptide; a peptide or protein from one of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, and Epstein-Barr virus.
39. The method of any one of the paragraphs 35-38, wherein the active moiety comprises a plurality of repeats of the disease-specific antigen.
40. The method of paragraph 39, wherein the plurality of the repeats of the disease-specific antigen is in the range of 2-50.
41. The method of paragraph 39, wherein the plurality of the repeats of the disease-specific antigen is in the range of 2-30.
42. The method of paragraph 39, wherein the plurality of the repeats of the disease-specific antigen is in the range of 3-20.
43. The method of any one of the paragraphs 39-42, wherein the plurality of the repeats of the disease-specific antigen is fused together.
44. The method of any one of the paragraphs 39-43, wherein the plurality of the repeats of the disease-specific antigen is arranged in a linear, branched, or circular manner.
45. The method of any one of the paragraphs 35-44, wherein the subject is a mammal.
46. The method of paragraph 45, wherein the mammal is a human.
47. The method of any one of the paragraphs 35-46, wherein the administering is systemic.
48. A composition comprising (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least two repeats of a disease-specific antigen.
49. The composition of paragraph 48, wherein the target receptor is selected from the group consisting of CD11c, DEC205/CD205, CD11b, CD206, CD209, Dectin-2, CD207, CD103, CD1d1, CD141/BDCA-1, CD68, CD1c/BDCA-1, and XCR1.
50. The composition of paragraph 48 or 49, wherein the disease-specific antigen is selected from the group consisting of a cancer antigen, a bacterial antigen, and a viral antigen.
51. The composition of any one of the paragraphs 48-50, wherein the disease specific antigen is 6-20 amino acids long.
52. The composition of any one of the paragraphs 48-51, wherein the disease-specific antigen is selected from the group consisting of cancer antigen 125; cancer antigen 15-3; cancer antigen 19-9; prostate cancer antigen 3; alphafetoprotein; carcinoembryonic antigen; epithelial tumor antigen; tyrosinase; a human Papillomavirus 16 peptide; a human P53 peptide; a human immunodeficiency virus peptide; an MUC-I human cancer antigen peptide; a peptide from proteins of MAGE gene family; a peptide from human tyrosinase protein; a Listeriolysin-O peptide; a P60 peptide; a MART-1 peptide; a BAGE-1 peptide; a P1A peptide; a Connexin gap junction derived peptide; a peptide or protein from one of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, and Epstein-Barr virus.
53. The composition of any one of the paragraphs 48-52, wherein the active moiety comprises a plurality of repeats of the disease-specific antigen.
54. The composition of paragraph 53, wherein the plurality of repeats of the disease-specific antigen is in the range of 2-50.
55. The composition of paragraph 53, wherein the plurality of repeats of the disease-specific antigen is in the range of 2-30.
56. The composition of paragraph 53, wherein the plurality of repeats of the disease-specific antigen is in the range of 3-20.
57. The composition of any one of the paragraphs 53-56, wherein the plurality of the disease-specific antigen is fused together.
58. The composition of any one of the paragraphs 53-57, wherein the plurality of the disease-specific antigen is arranged in a linear, branched, or circular manner.
59. The composition of any one of the paragraphs 48-58, further comprising a pharmaceutically-acceptable carrier or adjuvant.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology described herein is further illustrated by the following examples which in no way should be construed as being further limiting.

### Example 1

### Modified Anthrax Toxin for Delivery of Cell-Impermeable Therapeutic Agents

A DC-targeted ATx (anthrax toxin) epitope delivery system was created by combining mAT-DTR with a chimeric protein-antigen fusion, between LF_{N} and OVA₂₅₇₋₂₆₄ peptide (LF_{N}-OVA). This delivery system is referred to as mAT-DTR+LF_{N}-OVA. DTR and OVA₂₅₇₋₂₆₄ were chosen based on the availability of validated transgenic mouse models that either express DTR strictly on the surface of DCs (CD11c-DTR) or generate OVA-specific CTL responses (OT-I), respectively.

First, the magnitude of OVA-specific CTL responses in vitro and in vivo is characterized. Next, the ability of these fusions to stimulate protective immunity to a model bacterial pathogen is evaluated. Finally, results are confirmed in wild-type mice by creating a novel ATx epitope delivery system that targets the mouse DC receptor CD11c (termed mAT-αCD11c).

The receptor-targeted epitope delivery system described herein can be used to immunize against a wide array of bacterial, viral, and parasitic pathogens. In addition, several human and murine tumor antigens are described herein. Therefore, incorporation of these epitopes into this delivery system can also have applications in anti-cancer vaccines.

TABLE 2
Abbreviations

| Abbreviation | Description |
|---|---|
| LF_{N}-OVA | fusion of the N-terminal PA-binding domain of LF (LF_{N}) |
| | and OVA₂₅₇₋₂₆₄ peptide |
| LF_{N}-OVAx2 | fusion of the N-terminal PA-binding domain of LF (LF_{N}) |
| | and 2 OVA₂₅₇₋₂₆₄ peptides |
| LF_{N}-OVAx5 | fusion of the N-terminal PA-binding domain of LF (LF_{N}) |
| | and 5 OVA₂₅₇₋₂₆₄ peptides |
| LF_{N}-OVAx9 | fusion of the N-terminal PA-binding domain of LF (LF_{N}) |
| | and 9 OVA₂₅₇₋₂₆₄ peptides |
| wtAT | wild-type protective antigen of Anthrax toxin (AT) |
| mAT | untargeted mutant Anthrax toxin that lacks the native |
| | receptor-recognition domain |
| mAT-DTR | targeted mAT fused to the receptor-binding domain of |
| | Diphtheria toxin (DT) |
| mAT-CD11c | targeted mAT fused to the receptor-binding domain of |
| | CD11c expressed in DCs |
| OVA-mDT | OVA₂₅₇₋₂₆₄ fused to a non-toxic variant of Diphtheria |
| | toxin (DT) |
| Lm-OVA | Listeria monocytogenes genetically engineered to express |
| | OVA₂₅₇₋₂₆₄ peptide |

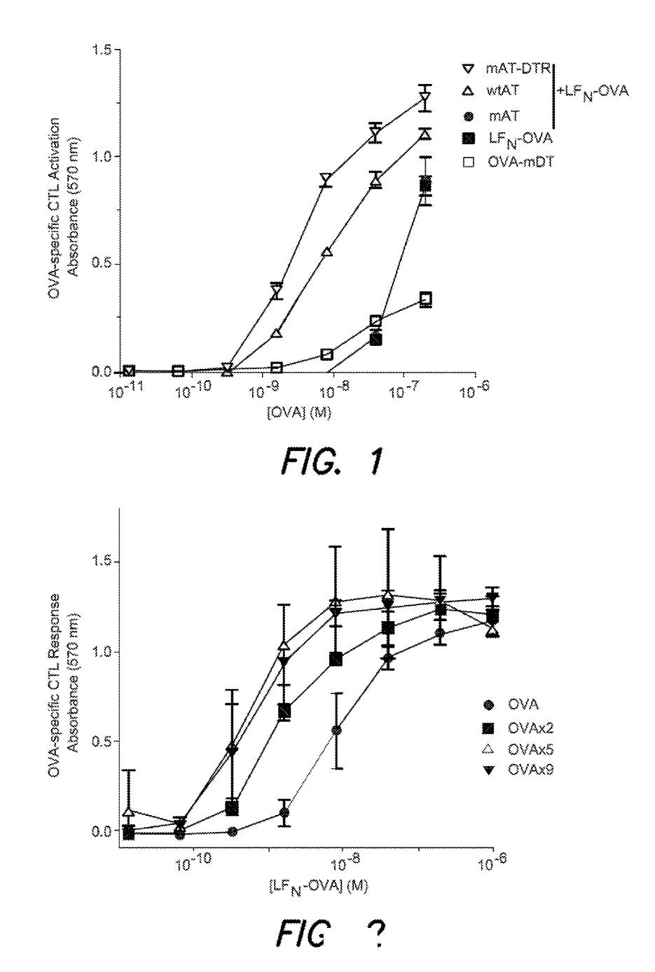

In vitro and in vivo results demonstrate that DC-targeted anthrax efficiently delivers OVA to DCs eliciting a robust OVA-specific CTL activation (FIGS. 1-3).

As shown in FIG. 1, the magnitude and kinetics of CTL activation was tested in vitro, by delivery of OVA (LF_{N}-OVA) into the cytosol of DCs. DCs were derived from bone-marrow of CD11c-DTR transgenic mice and treated with various concentrations of (i) mAT-DTR +LF_{N}-OVA, (ii) wtAT+LF_{N}-OVA, (iii) mAT+LF_{N}-OVA, (iv) LF_{N}-OVA and (v) OVA-mDT. Four hours later, B3Z T cells were added to DCs and B3Z T cell activation was determined by addition of CPRG. Delivery of OVA and CTL activation was compared between groups. The results demonstrated that in vitro mAT-DTR+LF_{N}-OVA generated enhanced OVA-specific CTL activation, compared to wtAT+LF_{N}-OVA, OVA-mDT or LF_{N}-OVA alone.

Using the same in vitro approach, the possibility of the ATx system described herein to deliver antigen repeats was tested in vitro, which may boost CTL activation. The results demonstrated that delivery of LF_{N}-OVAx2, LF_{N}-OVAx5 and LF_{N}-OVAx9 elicited a significantly stronger CTL response than the delivery of one single OVA peptide (see FIG. 2).

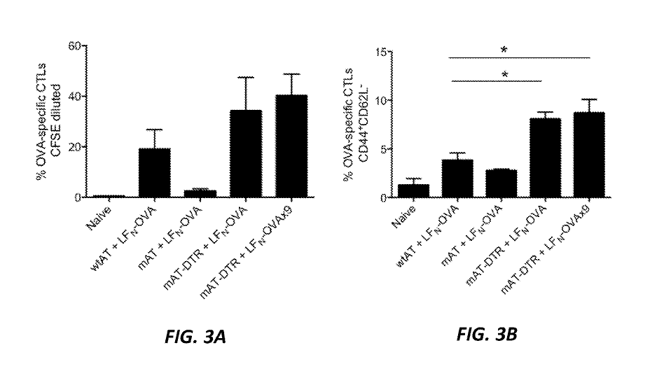

The ability of DC-targeted ATx (mAT-DTR) to deliver OVA antigen was evaluated in vivo. To test this, OVA-specific CTLs were isolated from OT-I mice (CD45.1⁺, TCRα2⁺ TCRβ5.1/5.2⁺), labeled ex vivo with CF SE and injected intravenously (i.v.) into CD11c-DTR mice (CD45.2⁺) (1.5×10⁶ OT-I CTLs/mouse). Twenty-four hours after OT-I CTL transfer, mice were left untreated or treated with (i) wtAT+LF_{N}-OVA, (ii) mAT+LF_{N}-OVA, (iii) mAT-DTR+LF_{N}-OVA or (iv) mAT-DTR+LF_{N}-OVAx9. Three days post-immunization, splenocytes were isolated and flow cytometry was used to measure OVA-specific CTL proliferation (CFSE dilution) and activation (CD44⁺ and CD62L^{low}). Consistent with in vitro results, proliferation and activation of OVA-specific CTLs were more robust when OVA was delivered by mAT-DTR (FIGS. 3A-3B). Interestingly, in this particular experiment, in vivo delivery of OVA repeats (mAT-DTR+LF_{N}-OVAx9) did not significantly improve CTL proliferation and activation. Without wishing to be bound by a theory, we consider that the reason to this was that the receptor, CD11c, is suspected to be at least partially internalized when the DC encounters an antigen, thus resulting in significant reduction of the receptor on the DC cell. Therefore, we suggest, that use of a more constantly present receptor, such as CD205 or XCR1 will provide improved in vivo results.

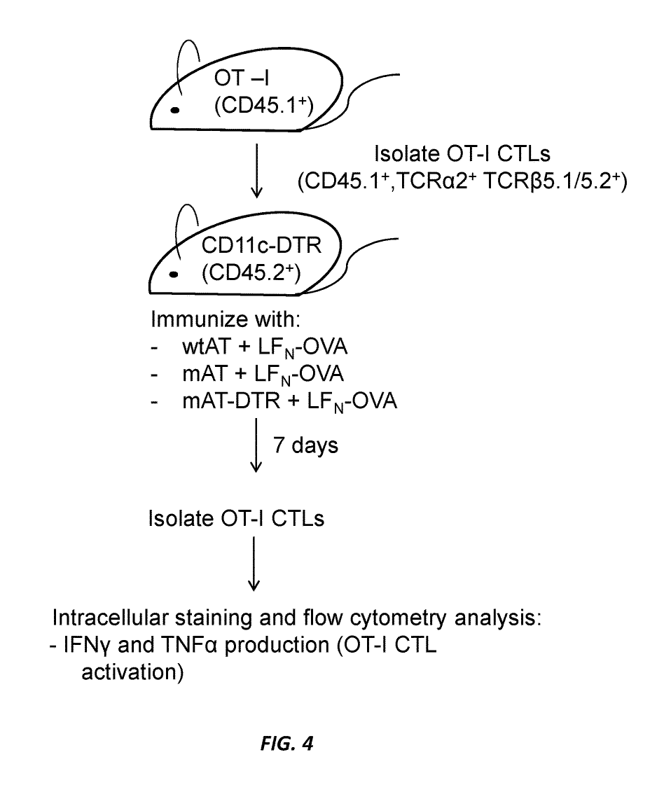

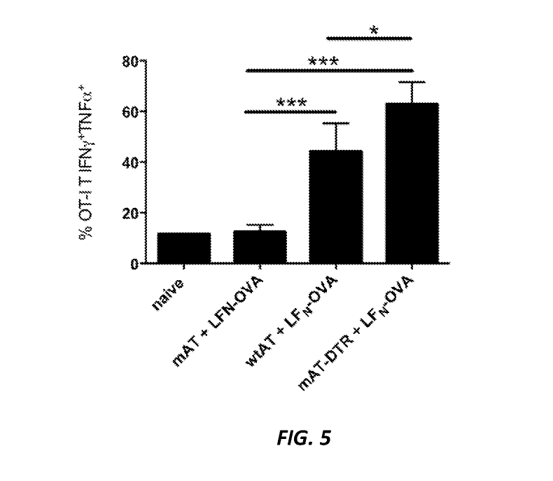

Experiments were performed to confirm that DC-targeted ATx system (mAT-DTR +LF_{N}-OVA) activates OT-I CTLs more robustly than untargeted ATx system by measuring IFNγ and TNFα. 1.5×10⁶ OT-I CTLs were transferred i.v. into CD11c-DTR mice. One day later, these mice were left untreated or treated i.p. with 30 pmol of (i) wtAT+LF_{N}-OVA, (ii) mAT+LF_{N}-OVA or (iii) mAT-DTR+LF_{N}-OVA. Seven days post-immunization, splenocytes were isolated from CD11c-DTR mice and IFNγ and TNFα produced by OVA-specific CTLs were measured by flow cytometry (FIG. 4). When OVA is delivered by the DC-targeted ATx system (mAT-DTR), OT-I CTLs produced significantly more amounts of IFNγ and TNFα than mice left untreated or treated with mAT+LF_{N}-OVA or wtAT+LF_{N}-OVA (FIG. 5).

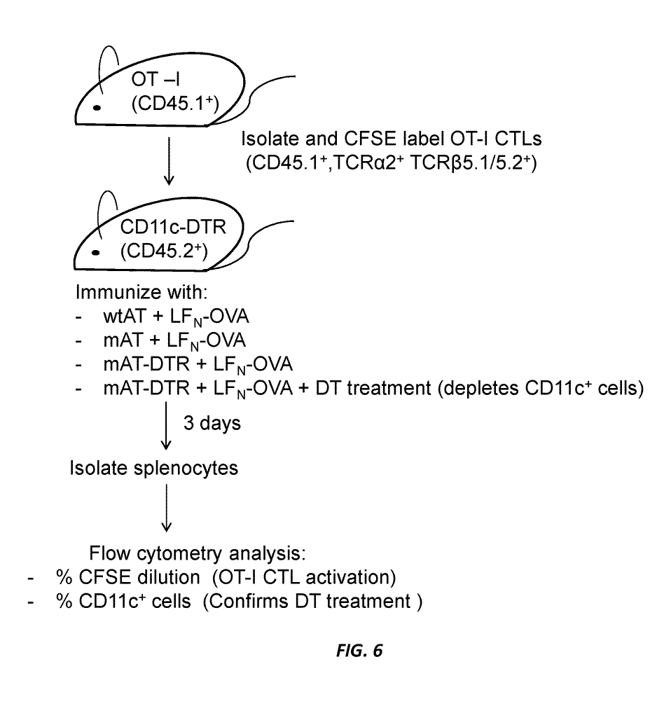

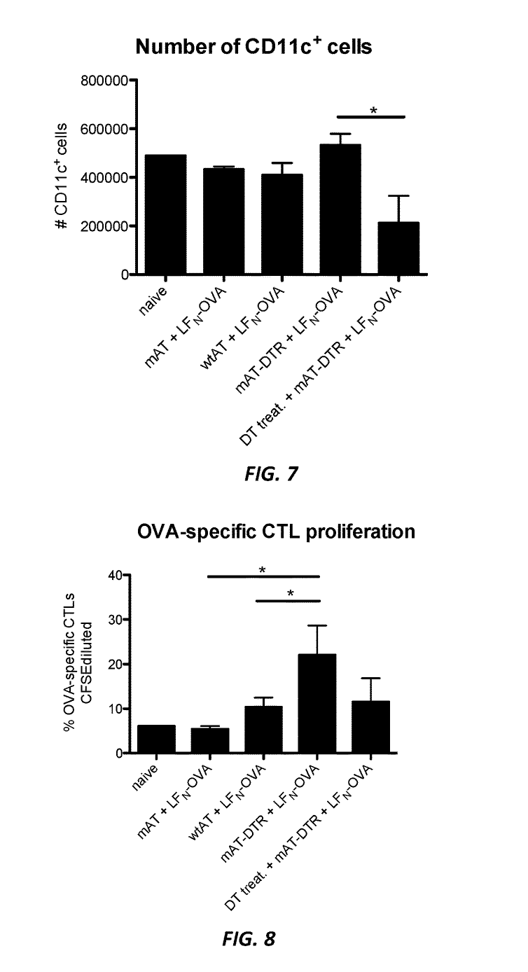

Experiments were performed to confirm that mAT-DTR specifically targets DTR expressed in DCs from CD11c-DTR mice. One group of 5 CD11c-DTR mice was treated i.p. with 25 ηg/g with diphtheria toxin (DT) to deplete all CD11c⁺ DTR⁺ cells. After DT treatment, CFSE-labeled OT-I CTLs (1.5×10⁶) were transferred into non-treated CD11c-DTR mice or DT-treated mice. The following day, non-treated mice were immunized i.p. with 30 ρmol of (i) mAT+LF_{N}-OVA , (ii) wtAT+LF_{N}-OVA or (iii) mAT-DTR+LF_{N}-OVA. DT-treated mice were immunized with 30 ρmol of mAT-DTR+LF_{N}-OVA. Three days after immunization, animals were sacrificed, and spleens were isolated and prepared into single cell suspensions. % of CD11c⁺ cells between DT-treated and non-treated mice was compared by flow cytometry (FIG. 6). The results demonstrate that DT treatment did not deplete all CD11c⁺ cells (FIG. 7). Even though a significant reduction of CD11c⁺ cells was observed in mice treated with DT, there was still a good amount of CD11c⁺ cells detected 4 days after DT treatment (FIG. 7). This is likely due to the high turnover of monocytes becoming CD11c⁺ DCs after immunization and one dose of DT administration might not be sufficient to deplete all CD11c⁺ cells for 4 days.

The % of OVA-specific CTLs that diluted CFSE was compared in mice treated or not treated with DT and immunized with mAT-DTR+LF_{N}-OVA. OT-I CTLs from DT treated mice proliferated less than non-treated mice (FIG. 8). This result is not statistically significant because depletion of CD11c⁺DTR⁺ cells was not very efficient. Even though DC depletion was not robust, when each mouse in the DT-treated group was analyzed, a positive correlation between amount of CD11c⁺ cells present and OT-I CTLs CFSE diluted was observed. Less CD11c⁺ cells present, less OT-I CTL proliferation was observed after immunization with mAT-DTR+LF_{N}-OVA (Table 3). These results demonstrate that DT treatment reduces CD11c⁺DTR⁺ cells from CD11c-DTR mice and therefore OVA delivery by mAT-DTR and consequent CTL activation is diminished. Therefore, mAT-DTR specifically targets the DTR expressed in CD11c-DTR DCs.

TABLE 3
mAT-DTR + LF_{N}-OVA + DT treatment

| # CD11c⁺ cells | % OVA-specific CTLs CFSE diluted |
|---|---|
| 29839 | 2.02 |
| 18294 | 5.35 |
| 473192 | 12.6 |
| 498759 | 31.6 |
| 36105 | 6.01 |

Experiments were performed to characterize OVA-specific memory T cell pool (hallmark of a robust vaccine after immunizing mice with different toxin delivery systems). Groups of 5 CD11c-DTR mice were immunized with 30 ρmol of either (i) wtAT+LF_{N}-OVA, (ii) mAT+LF_{N}-OVA, (iii) mAT-DTR+LF_{N}-OVA, or (iv) mAT-DTR+LF_{N}-OVAx9. Thirty days after immunization, OVA-specific memory T cell pool is analyzed by flow cytometry. Expression of memory T cell markers such as KLRG, CD127, CD44, CD62L, CCR7 are compared among groups. Since mAT-DTR+LF_{N}-OVA induced a potent CTL activation 3 and 7 days after immunization, the memory T cell pool in these mice should be stronger than the memory T cell pool generated by the other toxin delivery systems.

Experiments were performed to test whether DC-targeted ATx antigen delivery system can confer protection against live infection. Protective immunity against live bacterial infections requires strong CTL activation, proliferation and robust formation of a memory T cell pool against the pathogen. Experiments were performed to evaluate if immunization with mAT-DTR+LF_{N}-OVA can confer protection against live infection. To test this, groups of 5 CD11c-DTR mice were immunized i.v. with 30 ρmol of either (i) mAT+LF_{N}-OVA, (ii) wtAT-LF_{N}-OVA; (iii) mAT-DTR+LF_{N}-OVA (iv) mAT+LF_{N}-OVAx9, (iv) mAT-DTR+LF_{N}-OVAx9 or (iv) 5×10³c.f.u. of live Listeria monocytogenes genetically engineered to express the OVA₂₅₇₋₂₆₄ peptide (Lm-OVA). Thirty days later, immunized and non-immunized (control) mice are infected with an intravenous, sub-lethal dose of Listeria expressing OVA (10⁵ c.f.u.) (Lm-OVA). Three days post-infection, bacterial burden are determined by plating dilutions of spleen, liver and blood of infected mice on BHI agar. The number of OVA-specific CTLs stimulated by each immunization are quantified by staining with MHC-I pentamers folded with OVA₂₅₇₋₂₆₄ peptide. MHC-I pentamers are pentameric MHCI-I/peptide complexes containing a fluorescent tag that can be used in vitro to specifically bind T cells with that particular peptide/MHC-I specificity. These can be used to determine the frequency of OVA₂₅₇₋₂₆₄-specific CTLs over time by flow cytometry. In parallel, production of cytokines critical to mediate protective immunity (IFNγ and TNFα) that are produced by OVA-specific CTLs are assayed by flow cytometry and ELISPOT. Since previously described in vitro and in vivo results demonstrated that mAT-DTR+LF_{N}-OVA and mAT-DTR+LF_{N}-OVAx9 can induce a very robust CTL response compared to other delivery systems, these 2 toxin delivery systems should confer protection against Listeria similar to or better than the protection induced by a live infection.

Experiments were performed to evaluate the ATx antigen delivery system to target wild-type DCs and stimulate antigen-specific CTL responses. To demonstrate the potential of this delivery system to be translated to treat human disease, an mPA variant targeting the DC receptor CD11c (mAT-αCD11c) was created by fusing a validated anti-mouse CD11c single-chain antibody fragment to the C-terminus of mPA (FIG. 9). The resulting mPA-αCD11c fusion (mAT-αCD11c) was expressed and purified (data not shown). After creating this ATx fusion protein, the kinetics and magnitude of OVA-specific CTL responses in vitro as well as in vivo was characterized.

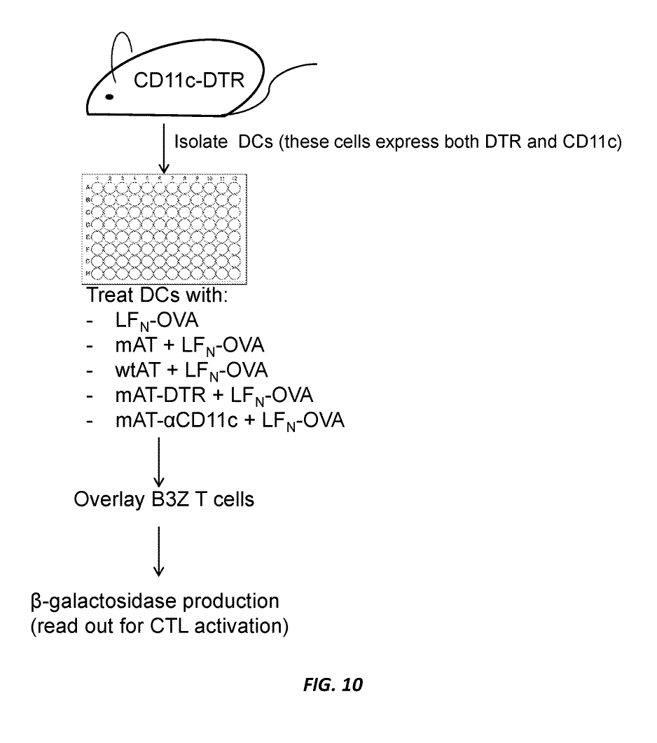

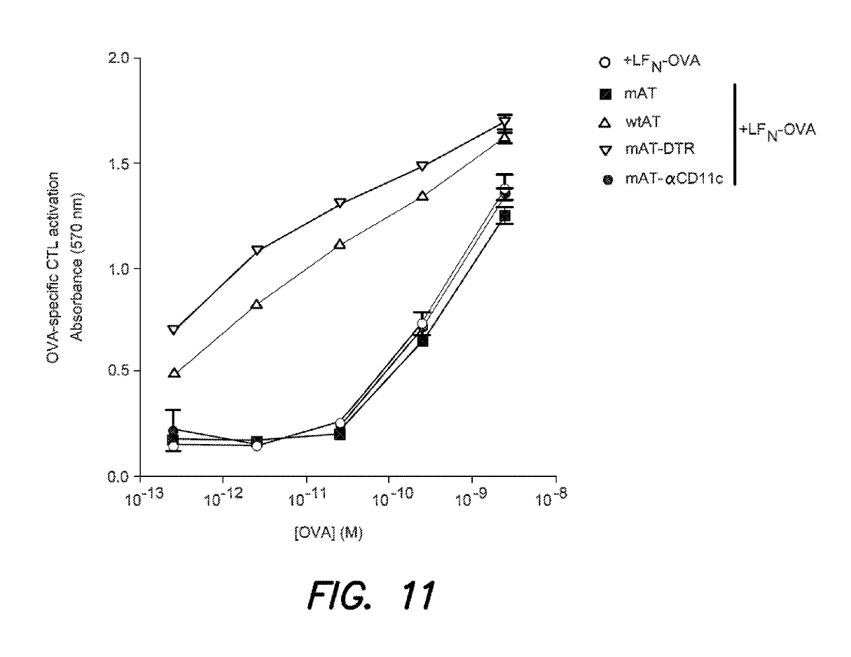

In vitro results: DCs were derived from CD11c-DTR mice. These cells express both CD11c and DTR at their surface. Seven days after differentiation, DCs were seeded in 96 well plates and exposed to defined concentrations of either (i) LE_{N}-OVA, (ii) mAT+LE_{N}-OVA, (iii) wtAT+LE_{N}-OVA, (iv) mAT-DTR+LE_{N}-OVA or (v) mAT-αCD11c+LE_{N}-OVA. B3Z T cells were added to each well and 24 hours after co-culture, the relative amount of B3Z T activation was determined by addition of CPRG. Delivery of OVA and consequent CTL activation was compared between all groups (FIG. 10). The experiment was done 3 times in triplicate. As expected, LE_{N}-OVA and mAT+LE_{N}-OVA induced minimal levels of CTL activation. mPA-DTR+LE_{N}-OVA elicited a strong OVA-specific CTL activation as compared to wtAT+LF_{N}-OVA. Surprisingly, mAT-αCD11c+LE_{N}-OVA induced minimal levels of CTL activation as compared to mAT-DTR+LE_{N}-OVA (FIG. 11). These in vitro results demonstrated that delivery of OVA antigen by mAT-αCD11c toxin and consequent CTL activation is inefficient in vitro.

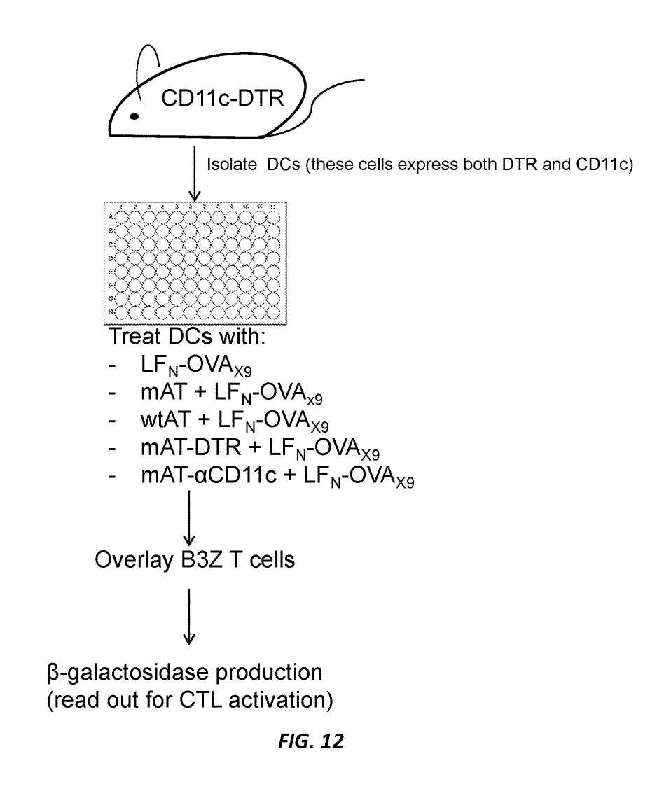

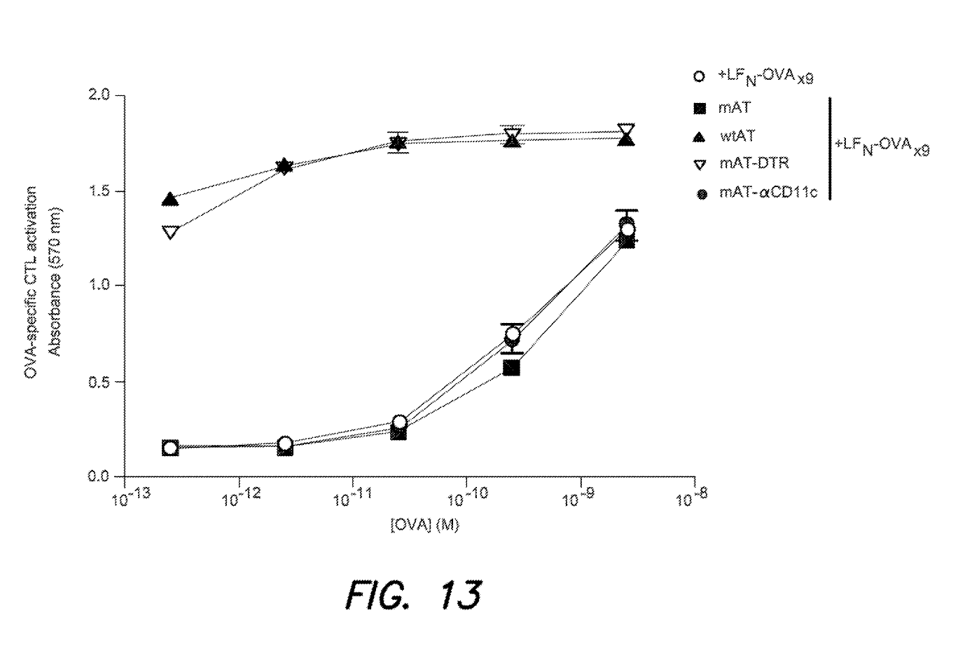

Whether delivery of OVA repeats by mAT-αCD11c could enhance OVA-specific CTL responses was tested. To test that, LF_{N}-OVAx9 was delivered to CD11c-DTR DCs by (i) mAT, (ii) wtAT, (iii) mAT-DTR or (iv) mAT-αCD11c. Using the same in vitro assay described above, the OVA-specific CTL responses were compared among groups (FIG. 12). As expected, wtAT+LF_{N}-OVAx9 and mAT-DTR+LFN-OVAx9 elicited a robust CTL response (FIG. 13). In contrast, CTL responses elicited by mAT-αCD11c+LF_{N}-OVAx9 were null (FIG. 13). Taken together, the in vitro results demonstrate that mAT-αCD11c+LE_{N}-OVA is either not targeting the CD11c⁺receptor or OVA is not delivered appropriately into the targeted cells.

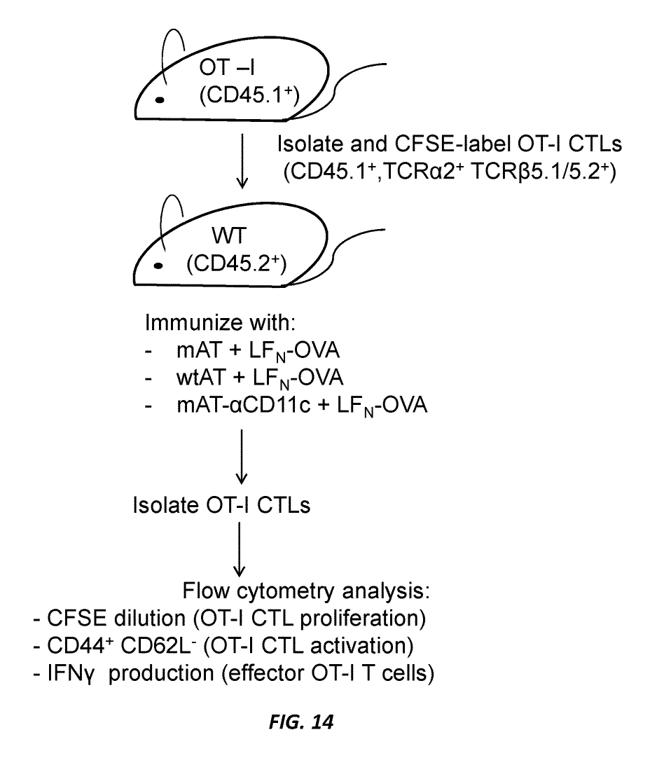

In vivo results: Despite that CD11c-targeted toxin fusion is inefficient at delivering OVA in vitro, the magnitude of CTL activation in vivo by DC-targeted mAT-αCD11c+LF_{N}-OVA was tested. To do that, 1.5×10⁶ OT-I CTLs (CD45.1⁺ TCRα⁺TCRβ5.1/5.2⁺) were isolated, CF SE labeled and transferred into C57BL/6 (WT) mice (CD45.2⁺). Twenty four hours after OT-I CTL transfer, groups of 5 WT mice were left untreated (naive) or immunized i.p. with 30 ρmol of (i) mAT+LE_{N}-OVA, (ii) wtAT+LE_{N}-OVA or mAT-αCD11c+LE_{N}-OVA. Three days after immunization, splenocytes were isolated and we measured OT-I CTL proliferation (CFSE dilution), activation (CD44⁺CD62L^{low}) and IFNγ produced by transferred OT-I CTLs using flow cytometry (FIG. 14). The experiment was done in triplicate.

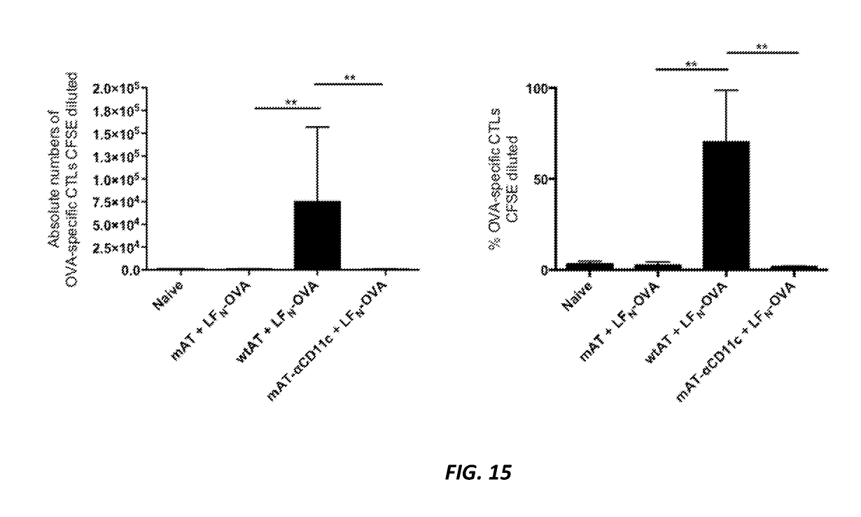

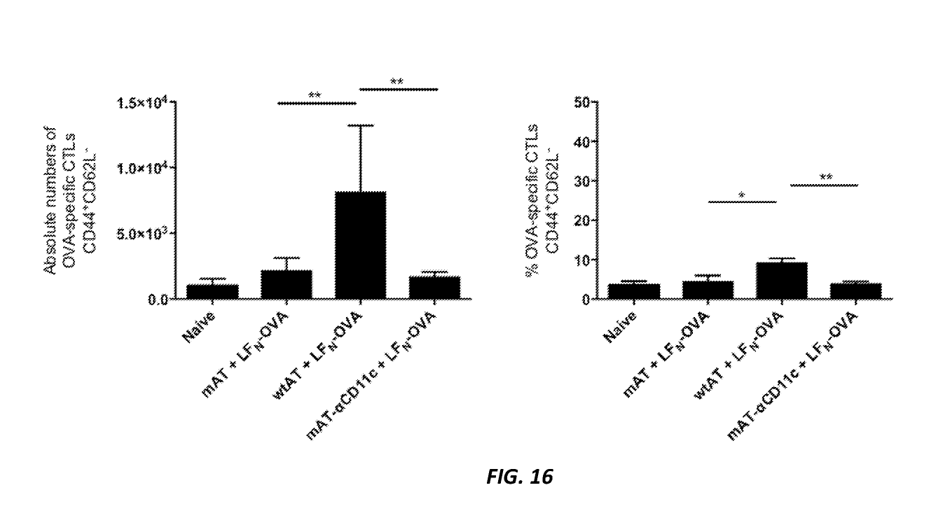

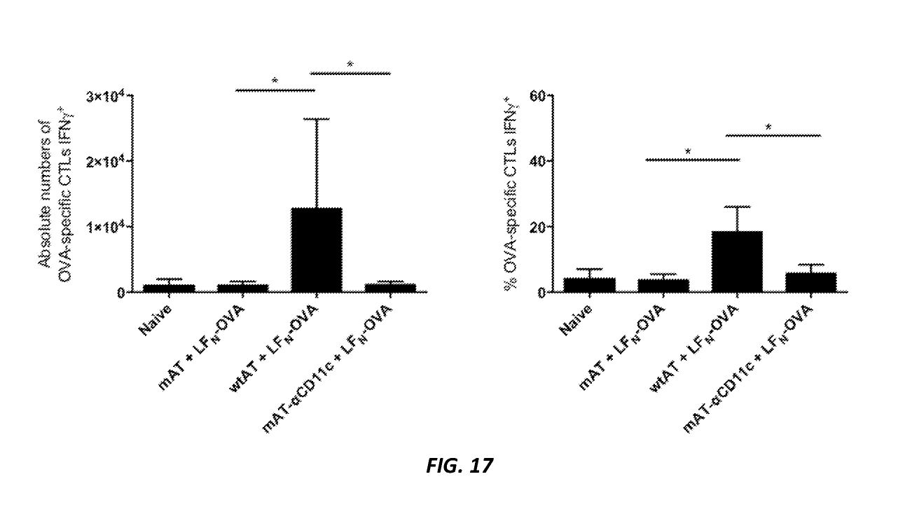

The results demonstrate that significant amounts of OT-I CTLs were activated (CD44⁺CD62L^{low}), proliferated (CF SE dilution) and produced IFNγ when mice were immunized with wtAT+LF_{N}-OVA, as compared to naive mice or mice treated with mAT+LF_{N}-OVA (FIGS. 15-17). However, in this experiment only small number of OT-I CTLs that were activated, that proliferated and that produced IFNγ were detected in mice immunized with mAT-αCD11c+LF_{N}-OVA (FIGS. 15-17). Taken together these in vitro and in vivo results demonstrate that delivery of OVA to DCs through the CD11c receptor elicits OVA-specific CTL response. However, the results can likely be significantly improved by use of a target receptor that is known to stay put on DCs during membrane remodeling at the time of their encounter with the antigen.

Thus, without wishing to be bound by theory, the low activation using the CD11c receptor as a target might be a result of: 1) DTR expression being higher than CD11c expression at the surface of CD11c-DTR DCs and therefore mAT-αCD11c toxin does not bind to CD11c as efficiently as mAT-DTR binds to DTR; and 2) a previous study has reported that CD11c is downregulated once DCs are exposed to TLR ligands and become activated (Singh-Jasuj a et al., 2013). Downregulation of CD11c in DCs upon activation, might interfere with mAT-αCD11c binding and consequent assembly of the toxin; 3) CD11c may not be the ideal receptor for targeting DCs.

Experiments can be performed to compare surface expression of DTR and CD11c in DCs. DCs are derived from CD11c-DTR mice. After DC differentiation (7 to 8 days), these cells are stained with antibodies against mouse CD11c (CD11c-PE) and against human DTR (hbEGF-APC). The expression levels of CD11c and DTR in DCs can be compared by flow cytometry. If DTR expression is higher than CD11c expression, which suggests that the moiety of mAT-DTR to DCs is better than mAT-αCD11c moiety to DCs.

Experiments can be performed to check whether CD11c is downregulated during DC activation. DCs are derived from CD11c-DTR mice. After differentiation, DCs are left untreated or exposed to different TLR agonists to induce DC activation (e.g. LPS (TLR4 ligand), CpG (TLR9 ligand), flagellin (TLR5 ligand)). Fifteen minutes, 1 hour, 5 hours, 12 hours and 24 hours after treatment, DCs are collected and stained with antibodies against CD11c (CD11c-PE) and DTR (hbEGF-APC). Using flow cytometry, the expression levels of CD11c between activated and non-activated DCs can be compared. The expression of CD11c and DTR at the surface of activated DCs can also be compared. If CD11c is downregulated after exposure to different TLR ligands but DTR expression remains the same, that might explain the difference obtained when OVA is delivered by mAT-αCD11 c as compared to mAT-DTR.

Experiments can be performed to engineer ATx to target a different receptor exclusively expressed in DCs. It is possible that CD11c is not the ideal receptor for targeting DCs. If this is the case, an ATx fusion can be engineered to bind to other receptors exclusively expressed in DCs. One such receptor is DEC-205 (Demangel et al., 2004, Molecular Immunology). DEC-205 is a C-type lectin receptor expressed by both mouse DCs and some human DC subsets. A validated anti-mouse DEC 205 single-chain antibody fragment (scNLDC) can be fused to the C terminus of mPA, creating a mAT-αDEC205 toxin fusion. The mAT-αDEC205 fusion can be expressed and purified using the same methods used to express and purify the mAT-DTR fusion. The kinetics and magnitude of OVA-specific CTL responses in vitro and in vivo can be characterized, in response to mAT-αDEC205+LF_{N}-OVA. Delivery of OVA to DCs expressing DEC-205 can be further improved by administering an additional stimulus to trigger DC maturation, such as anti-DC40 (Demangel et al., 2004, Molecular Immunology; Hawigger et al., 2001, J. Exp. Med).

XCR1 is a chemokine receptor exclusively expressed on murine and human cross-presenting DCs. XCR1 is another potential candidate to target DCs (Harthung et al., 2015, J. of Immunology).

It would be optimal to elicit and robust CTL response as the more robust CTL responses can improve not only efficient clearance of pathogens but also clearance of some tumors. EG7 cells are mouse thymoma EL4 cells stably transfected with the complementary DNA of chicken ovalbumin and thus express SIINFEKL epitopes as a unique antigen. This tumor model can allow the evaluation of the magnitude of CTL specific responses against the tumor once OVA is delivered by the DC-targeted ATx delivery platform.

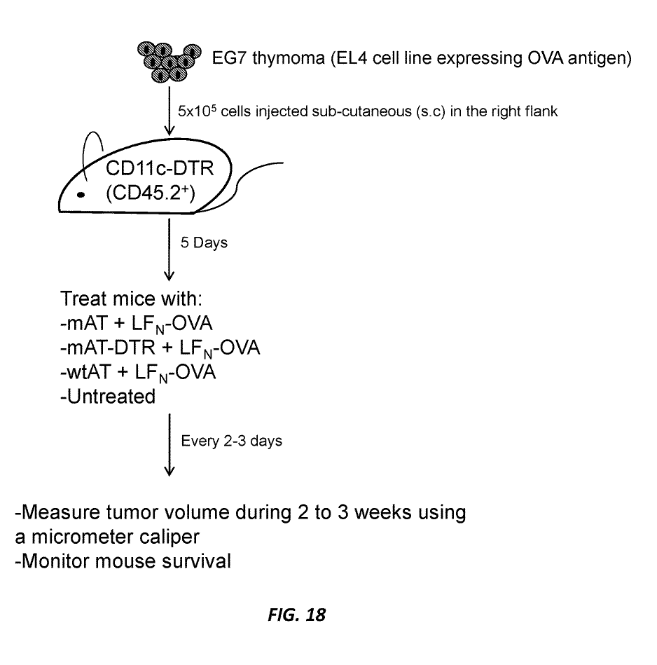

Experiments can be performed to test the use of DC-targeted toxin as a therapeutic strategy against tumors. 5×10⁵ EG7 thymoma cells (EL4 cell line expressing OVA antigen) are injected sub-cutaneously (s.c) in the right flank of CD11c-DTR mice. After letting the tumor cells grow for 5 days, CD11c-DTR mice are treated with either i) mAT+LF_{N}-OVA ii) mAT-DTR+LF_{N}-OVA or iii) wtAT+LF_{N}-OVA. As control, a group of mice untreated is left untreated. Every 2 to 3 days measure tumor growth is measured using a micrometer caliper and mouse survival is monitored for 30 days (FIG. 18). mAT-DTR+LF_{N}-OVA should elicit a very robust OVA-specific CTL response that is sufficient to control tumor growth. wtAT+LF_{N}-OVA treatment should also limit tumor growth to a certain extent. In contrast, the tumor development should be similar in mice treated with mAT +LF_{N}-OVA and untreated mice.

To confirm that this therapeutic strategy is antigen specific, CD11c-DTR mice can be injected with EL4 cells. These cells can induce tumors exactly like EG7 cells but they don't express the OVA antigen, therefore treatments with mAT-DTR+LF_{N}-OVA or wtAT+LF_{N}-OVA should not prevent tumor growth.

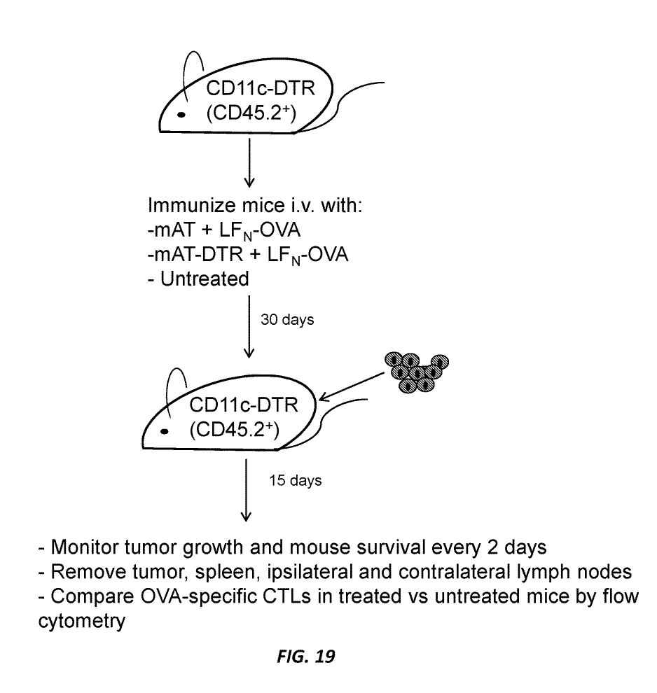

Experiments can be performed to test the use of DC-targeted toxin as a prophylactic strategy against tumors. CD11c-DTR mice are immunized i.v. with i) mAT+LF_{N}-OVA, ii) mAT-DTR+LF_{N}-OVA or iii) left untreated. The mice are left to rest for 30 days to allow formation of a memory T cell pool against OVA. One month after immunization, 5×10⁵ EG7 cells are injected s.c. in the right flank of CD11c-DTR mice. Tumor growth is monitored every 2 days in each group of mice. Fifteen days after EG7 cell injection, the mice is sacrificed, tumor, spleens, isilateral lymph nodes (tumor-draining lymph node), and contralateral lymph nodes (non-draining lymph node) are removed, and the OVA-specific CTL responses in each group can be compared by flow cytometry (FIG. 19). mAT-DTR+LF_{N}-OVA-immunized mice should have smaller tumors than non-immunized mice. There should be more OVA-specific CTL infiltration in the spleen and in the ipsilateral lymph nodes of the mAT-DTR+LF_{N}-OVA immunized mice, as compared to the spleen and lymph nodes of mAT+LF_{N}-OVA treated mice or untreated mice.

This platform can be used alone or in combination with other therapeutic strategies to prevent and combat cancer in humans.

### Example 2

### Alternative Approaches to Use the DC-Targeted Toxin Antigen Delivery Platform

Robust CTL responses are important not only for efficient clearance of pathogens but also for clearance of some tumors. Described herein is the use of the ATx antigen delivery platform to induce antigen-specific responses against tumors.

The E.G7-OVA lymphoma cell line was used in the experiments described herein. E.G7-OVA was derived in 1988 from the C57BL/6 (H-2b) mouse lymphoma cell line EL4. The EL4 cells were transfected by electroporation with the plasmid pAc-neo-OVA which carries a complete copy of chicken ovalbumin (OVA) mRNA and the neomycin (G418) resistance gene. This cell line expresses SIINFEKL epitopes as a unique antigen. This is a quite well described tumor model that permits evaluation of the magnitude of CTL specific responses against the tumor once OVA is delivered by the DC-targeted ATx delivery platform,

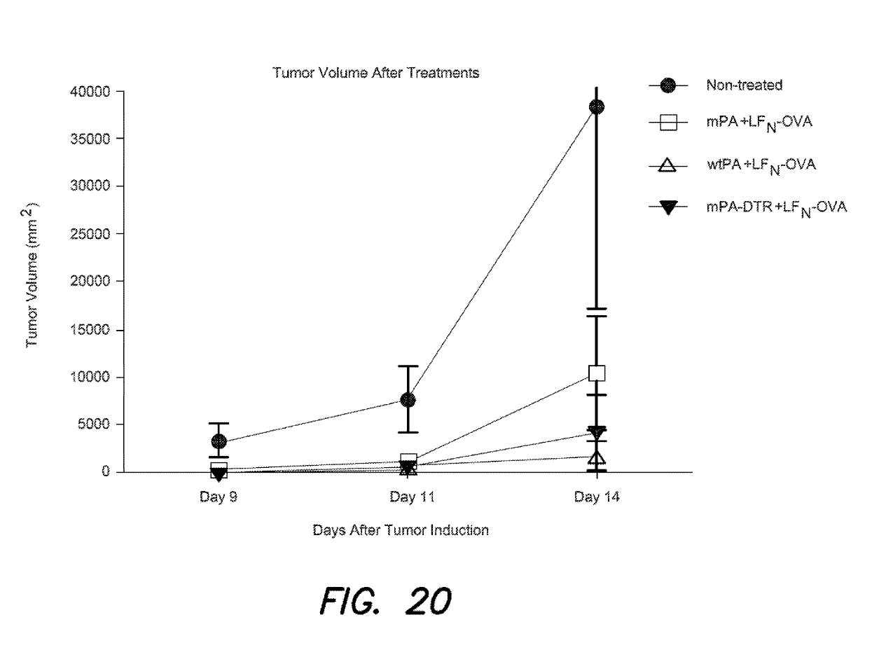

First, the use of DC-targeted toxin as a therapeutic strategy against tumors was tested. To do that, 5×10⁵ E.G7-OVA were injected sub-cutaneously (s.c) in the right flank of CD11c-DTR mice. After letting the tumor cells grow for 5 days, CD11c-DTR mice were treated with either i) mPA-DTR+LF_{N}-OVA ii) wtPA+LF_{N}-OVA, iii) mPA+LF_{N}-OVA or left untreated (FIG. 18). Every 2 to 3 days tumor growth was measured using a micrometer caliper and mouse survival was monitored for 15 days (FIG. 20).

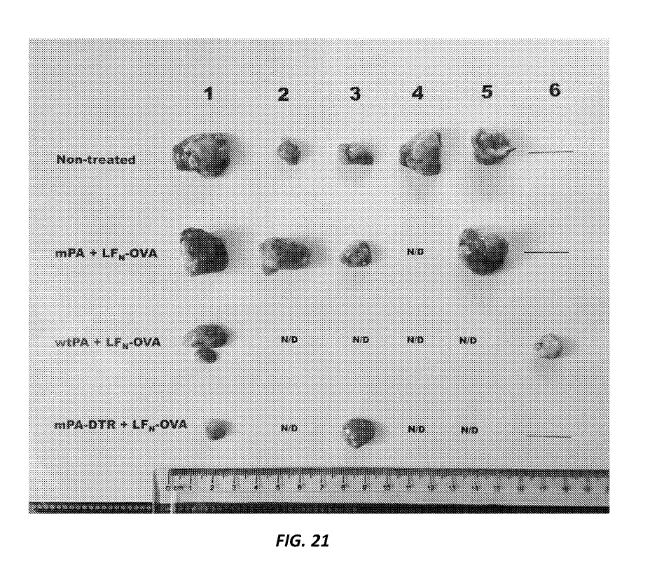

Mice treated with mPA-DTR+U_{N}-OVA as well as mice treated with wtPA+LF_{N}-OVA did not develop tumors or had significantly smaller tumors than mice treated with mPA+LF_{N}-OVA or mice left untreated (FIGS. 20 and 21). These results demonstrate that delivery of LF_{N}-OVA by either wtPA or mPA-DTR induces a CTL response that is sufficient to inhibit growth of tumors expressing OVA antigen.

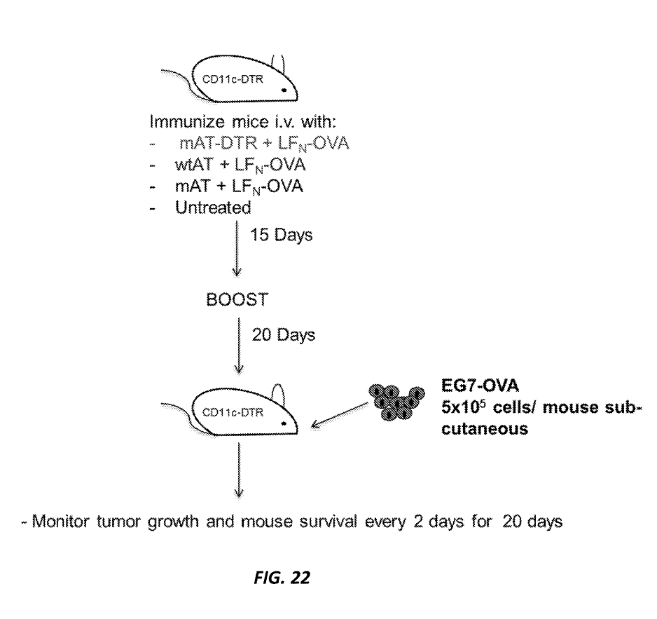

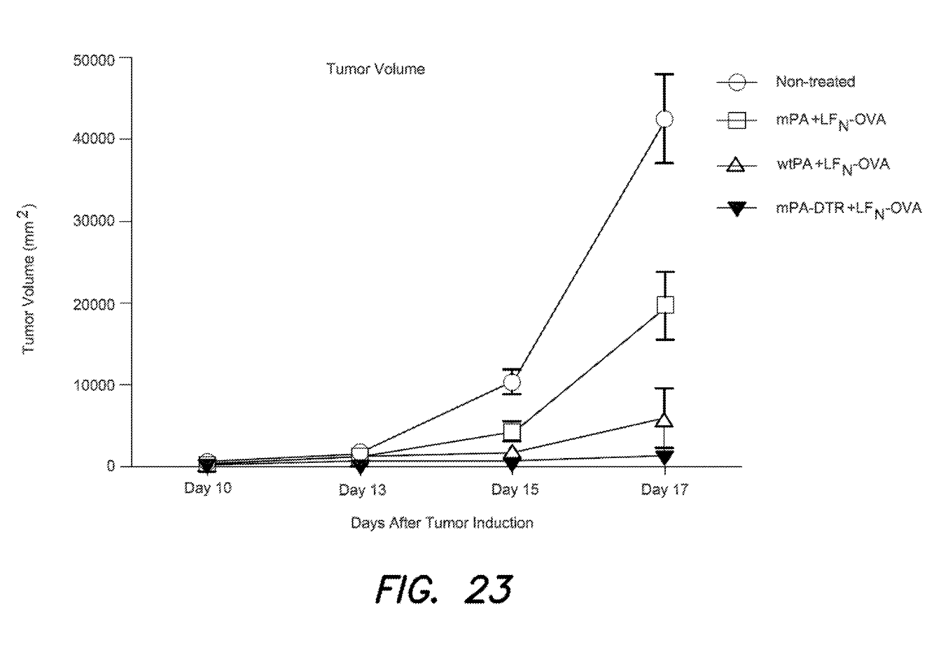

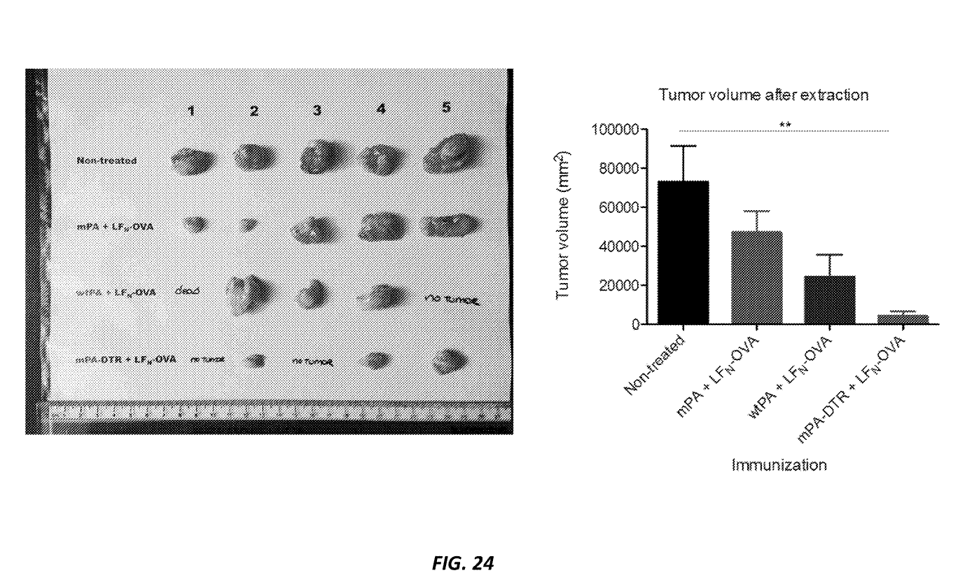

Next, the use of DC-targeted toxin as a prophylactic strategy against tumors was tested. To do that, CD11c-DTR mice were immunized intravenously with i) mPA-DTR+LF_{N}-OVA, ii) wtPA+mPA+LF_{N}-OVA or left untreated. Fifteen days later, mice were boosted and allowed to rest for 20 days to allow formation of a memory T cell pool against OVA. CD11c-DTR mice were then injected sub-cutaneously in the right flank with 5×10⁵E,G7-OVA and tumor growth as well as mouse survival was monitored every 2-3 days for 20 days (FIG. 22). Tumor growth was slower in mice immunized with mPA-DTR+LF_{N}-OVA or immunized with wtPA+LF_{N}-OVA as compared to untreated mice or mice treated with mPA+LF_{N}-OVA (FIG. 23). Twenty days after tumor induction, tumors were extracted and tumor volume was measured ex vivo. Mice immunized with mPA+LF_{N}-OVA had tumors with sizes comparable to tumors observed in non-immunized mice. In contrast, mPA-DTR+LF_{N}-OVA-immunized mice had significantly smaller tumors than non-immunized mice. wtPA+LF_{N}-OVA-immunized mice also had smaller tumors as compared to non-immunized mice or mice immunized with mPA+LF_{N}-OVA. Taken together, these results demonstrate that immunization of mice with mPA-DTR+LF_{N}-OVA as well as wtP+LF_{N}-OVA can prevent the growth of tumors expressing OVA antigen.

These experiments demonstrate that DC-targeted ATx platform can be used as an antigen-specific therapeutic and prophylactic strategy against tumors. It is contemplated herein that other known tumor-specific antigens can also be fused to LF_{N} and expected to work similarly. This platform can be used alone or in combination with other therapeutic strategies to prevent and combat cancer in humans.

1. A method of delivering a disease-specific antigen into a dendritic cell, the method comprising contacting the dendritic cell with a composition comprising (a) a native-receptor-ablated anthrax toxin protective antigen (PA) fused to a receptor-binding moiety specific for a target receptor on the dendritic cell and (b) a lethal factor (LF) or a fragment thereof fused to an active moiety comprising at least one repeat of the disease-specific antigen.

2. The method of claim 1, wherein the target receptor is selected from the group consisting of CD11c, DEC205/CD205, CD11b, CD206, CD209, Dectin-2, CD207, CD103, CD1d1, CD141/BDCA-1, CD68, CD1c/BDCA-1, and XCR1.

3. The method of claim 1, wherein the disease-specific antigen is selected from the group consisting of a cancer antigen, a bacterial antigen, and a viral antigen.

4. The method of claim 1, wherein the disease-specific antigen is selected from the group consisting of: cancer antigen 125; cancer antigen 15-3; cancer antigen 19-9; prostate cancer antigen 3; alphafetoprotein; carcinoembryonic antigen; epithelial tumor antigen; tyrosinase; a human Papillomavirus 16 peptide; a human P53 peptide; a human immunodeficiency virus peptide; an MUC-I human cancer antigen peptide; a peptide from proteins of MAGE gene family; a peptide from human tyrosinase protein; a Listeriolysin-O peptide; a P60 peptide; a MART-1 peptide; a BAGE-1 peptide; a P1A peptide; a Connexin gap junction derived peptide; a peptide or protein from one of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, and Epstein-Barr virus.

5. The method of claim 1, wherein the active moiety comprises a plurality of repeats of the disease-specific antigen.

6.-12. (canceled)

13. The method of claim 1, wherein the contacting is performed in vitro.

14. The method of claim 1, wherein the contacting is performed in vivo.

15. A method of inducing an immune response in a subject, the method comprising administering to the subject a composition comprising (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least one repeat of a disease-specific antigen.

16. The method of claim 15, wherein the immune response is a protective immune response.

17. The method of claim 15, wherein the target receptor is selected from the group consisting of CD11c, DEC205/CD205, CD11b, CD206, CD209, Dectin-2, CD207, CD103, CD1d1, CD141/BDCA-1, CD68, CD1c/BDCA-1, and XCR1.

18. The method of claim 15, wherein the disease-specific antigen is selected from the group consisting of a cancer antigen, a bacterial antigen, and a viral antigen.

19. The method of claim 18, wherein the induced immune response is against a cancer or against a bacterial infection or against a viral infection.

20. (canceled)

21. (canceled)

22. The method of claim 15, wherein the disease-specific antigen is selected from the group consisting of: cancer antigen 125; cancer antigen 15-3; cancer antigen 19-9; prostate cancer antigen 3; alphafetoprotein; carcinoembryonic antigen; epithelial tumor antigen; tyrosinase; a human Papillomavirus 16 peptide; a human P53 peptide; a human immunodeficiency virus peptide; an MUC-I human cancer antigen peptide; a peptide from proteins of MAGE gene family; a peptide from human tyrosinase protein; a Listeriolysin-O peptide; a P60 peptide; a MART-1 peptide; a BAGE-1 peptide; a P1A peptide; a Connexin gap junction derived peptide; a peptide or protein from one of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, and Epstein-Barr virus.

23. The method of claim 15, wherein the active moiety comprises a plurality of repeats of the disease-specific antigen.

24.-28. (canceled)

29. The method of claim 15, wherein the active moiety comprises at least two types of disease-specific antigen.

30.-34. (canceled)

35. A method of enhancing cytotoxic-T lymphocyte (CTL) activation in a subject, the method comprising administering to the subject a composition comprising (a) a native-receptor-ablated anthrax toxin protective antigen (PA) fused to a receptor-binding moiety specific for a target receptor on a dendritic cell and (b) a lethal factor (LF) or a fragment thereof fused to an active moiety comprising at least one repeat of a disease-specific antigen.

36. The method of claim 35, wherein the target receptor is selected from the group consisting of CD11c, DEC205/CD205, CD11b, CD206, CD209, Dectin-2, CD207, CD103, CD1d1, CD141/BDCA-1, CD68, CD1c/BDCA-1, and XCR1.

37. The method of claim 35, wherein the disease-specific antigen is selected from the group consisting of a cancer antigen, a bacterial antigen, and a viral antigen.

38. The method of claim 35, wherein the disease-specific antigen is selected from the group consisting of: cancer antigen 125; cancer antigen 15-3; cancer antigen 19-9; prostate cancer antigen 3; alphafetoprotein; carcinoembryonic antigen; epithelial tumor antigen; tyrosinase; a human Papillomavirus 16 peptide; a human P53 peptide; a human immunodeficiency virus peptide; an MUC-I human cancer antigen peptide; a peptide from proteins of MAGE gene family; a peptide from human tyrosinase protein; a Listeriolysin-O peptide; a P60 peptide; a MART-1 peptide; a BAGE-1 peptide; a P1A peptide; a Connexin gap junction derived peptide; a peptide or protein from one of the following pathogens: Cytomegalovirus, Hepatitis B, Human Herpes Virus 1-5, Rabies Virus, Meassles Virus, Mumps Virus, Rubella Virus, Shigella, Mycobacterium tuberculosis and avium, Salmonella typhi and typhimurium, HTLV-I, HTLV-II, Varicella zoster, Variola, Polio, Yellow Fever, Encephalitis viruses, and Epstein-Barr virus.

39. The method of claim 35, wherein the active moiety comprises a plurality of repeats of the disease-specific antigen.

40.-59. (canceled)
